SEQUENCE LISTING (10 sequences)

SEQ ID NO: 1 - DNA, 4882 bp, Homo sapiens
ggcgttacgggatgaattaacggcgggttccgcacggaggttgtgacccctacggagccccagcttgcccacgcaccccactcggcgtcgcgcggcgtgccctgcttgtcacagaatcatggtgtcatggaaagggatttactttatactgactctgttttggggaagcttttttggaagcattttcatactgagtccctttttacctttgatgtttgtaaacccatcttggtatcgctggatcaacaaccgccttgtggcaacatggctcaccctacctgtggcattattggagaccatgtttggtgtaaaagtgattataactggggatgcatttgttcctggagaaagaagtgtcattatcatgaaccatcggacaagaatggactggatgttcctgtggaattgcctgatgcgatatagctacctcagattggagaaaatttgcctcaaagcgagtctcaaaggtgttcctggatttggttgggccatgcaggctgctgcctatatcttcattcataggaaatggaaggatgacaagagccatttcgaagacatgattgattacttttgtgatattcacgaaccacttcaactcctcatattcccagaagggactgatctcacagaaaacagcaagtctcgaagtaatgcatttgctgaaaaaaatggacttcagaaatatgaatatgttttacatccaagaactacaggctttacttttgtggtagaccgtctaagagaaggtaagaaccttgatgctgtccatgatatcactgtggcgtatcctcacaacattcctcaatcagagaagcacctcctccaaggagactttcccagggaaatccactttcacgtccaccggtatccaatagacaccctccccacatccaaggaggaccttcaactctggtgccacaaacggtgggaagagaaagaagagaggctgcgttccttctatcaaggggagaagaatttttattttaccggacagagtgtcattccaccttgcaagtctgaactcagggtccttgtggtcaaattgctctctatactgtattggaccctgttcagccctgcaatgtgcctactcatatatttgtacagtcttgttaagtggtattttataatcaccattgtaatctttgtgctgcaagagagaatatttggtggactggagatcatagaacttgcatgttaccgacttttacacaaacagccacatttaaattcaaagaaaaatgagtaagattataaggtttgccatgtgaaaacctagagcatattttggaaatgttctaaacctttctaagctcagatgcatttttgcatgactatgtcgaatatttcttactgccatcattatttgttaaagatattttgcacttaattttgtgggaaaaatattgctacaattttttttaatctctgaatgtaatttcgatactgtgtacatagcagggagtgatcggggtgaaataacttgggccagaatattattaaacaatcatcaggcttttagcgacaaggaacacacacatttttcttaaaggcccaatcctaacagactccccgtaccagaggcagatctggaactcgtcaacaacagcccggatcacatgctgttactggactgcccaggtcacctcattgaggtgagcgctgtccctccaggtactcaggcaggtggtgcccaggctccctcccttggcgtccttcttcctgcgccaaacccatctcatccttaagatcagaaaatggaaccccctttgtcgtgaaaccgatcaagcattttgacaggaagcccttcattattcataacattgtatgctgaccaagacgtcagaagaccacctgtgcccttttctctcgtggaagataaaacctggactcagggagcaaccacaggctctccactttgaaactttagtttgaattttaaaagataggaagtaatacagtttaagttcctttctcacactcagtattgcatgcttagtgctggttttcttacaacattttggcaggtgtagctttttctgttaaaaatggtatacatgaagtcacatacttttttaaaggaacactcggaacattctgtcattccagtcagaaactgtcttttgaaatatttctttacaacctaccaaaaaaggaaggttgttttttctacatgtgcttgggtatctaacatctaccattaaaacaacaaatggcttgcagacagggcagtaatggtttatcctaaattactcaaccccttgtagccttgacaaattttaccttaaaaccaaaatgaaacacaaaaattaatccttaataatgatagcaagtgatctttctttttagttttagccttcctttttcaatagtaatatttaaacccacttttgaccaattgtttgcccaaatattcttgtcatttggagtcagtggaaaatccagcacaccaagcaccagtcttcttctgaggcaaaagaaaagtgttgtcattttcactctgttggagctgcacacttttttttctttttttttttcttttttttttgtcatgtaagaaggatgctggtcagagctgcagaaaatatgaggcaattaaaagtctttagctgttagcaaacctgttagttttacttctgcattgaaccagcctcagaagctacttactgctttatgtactctttgggcattaatgccttctctgtaattatatctcgtttttgcttgacagtgacctacccagtaattgcatcgtgcattgccatgaaaggtaaacacattgtgaactgaacttaccaagcagattctgtgagaaagcactggttgaggctgaacactgttgacacatcatttttattggaagagtattaactggtgcctcttctgaaacacaccaacccatattcctctgctcccccaaagctgtttctgatcctgctgggagcaactaactagttattatgcacatctgctccagacccagctctttaacttcatggttttacagcttgttttttctttttcttttcttttcttttttttttttttaaaaagcacctttttttgttgtttccccttaataaaagaggtttctaatttatgtttctctaagatttcctttggttgtatttagaaacacaaatagttttataatcaggattgcatttgtgctgggtaaaagaggagagctgtgctttctaccccaaagttgttggtaatggagccggtctgcccttcctacaaactcaagaggctcattgttcaaggtgtaacaacatttaattgcatgtcctctaacgctttgaaacctttagagggaagatcctcattttttacaattttgttccttttcatcatagaaaacatgtttaagataaaatacaaactttacctaccccaaattcataaagtacatttagtgctgtatagcactaaaacttagagatacagacactgtacttactttttaagaattagagacggtaactccaaaaataattgtccttttttctttcttttttttttttttacaataaggctcttgaaaattgtcattacttgtgttttcctatacattcatctgtgtgaaagcctttttcttctttgatttaaaaaaattaactatacagttaatggtttagaacttagaactacttagaattaatgctaaagtgtcagtaagaaattaatttagcttcaataattgtgactggcctcaggaattctcccttccacacctgcccacctcacctcacctcacctcaccgcaccgcaccgcaccagagccagagcagctgcttgtctgcagcaggacacagttcctacatacgtttcagttctttcatggtaagctcaatggactttgaattgtttacagtgctgtatgtccaattgttaaatgtaccattctgaacgatgttaaagcaagtgtggtttatttatggcatgaaccatgtaacttgaaatatgaacttacaaggaggggcactcattaggtaacaagttcttacaccaaacttccttgatgaaataagccaaaataatcctaaaattcattagaagaacttgataaaagactcaaataaatgttagaaagagcccataattttaggactcctataaaattcttccttgtttgttaatgctattaaaactcagattcaagggaaataccagcttccacttgagtcactttgaaatagttaattcaacagcaccatgttagaaatatattggcagccaagactctgaactctgcagaaacatttgtttcacccagacttcaaactctagccctgactatgatgcccctgtgtgcatttacaataaagactccaacggaggacctgttggtgttaaattgtttacatttctttatacaaataatgtgctttcagtgccttagttacagctccctttctgtttcttgttccaaggattctgtagtatgtaatgtgtttcttaggtaaagtctctttttgctactgaaagggaaatggtctctaaacactggtcactgtagcaggtaaacactactctaacgtggagaaatgagcttcatgctgaggtagtggttgccttagagctgttatttatgctgtagaaaacgaaaatggttttgctacctgataagcttcagattagatatagccctaaagttatcctgtacctgcattaaattttcattttagaagagaatcttggttttggtaattcatttttttttaagatcattatgtgcaaaacaccaagttttaaaaataagtcacagaatggccttagttttcaatgtctgatggtatatttgtgagttgtgttatctgtaatatacatcccagaataaaggagtgaattaaatagtttaaaaaaaaaaaaaaaaaaa

SEQ ID NO: 2 - protein, 376 aa, Homo sapiens
MVSWKGIYFILTLFWGSFFGSIFILSPFLPLMFVNPSWYRWINNRLVATWLTLPVALLETMFGVKVIITGDAFVPGERSVIIMNHRTRMDWMFLWNCLMRYSYLRLEKICLKASLKGVPGFGWAMQAAAYIFIHRKWKDDKSHFEDMIDYFCDIHEPLQLLIFPEGTDLTENSKSRSNAFAEKNGLQKYEYVLHPRTTGFTFVVDRLREGKNLDAVHDITVAYPHNIPQSEKHLLQGDFPREIHFHVHRYPIDTLPTSKEDLQLWCHKRWEEKEERLRSFYQGEKNFYFTGQSVIPPCKSELRVLVVKLLSILYWTLFSPAMCLLIYLYSLVKWYFIITIVIFVLQERIFGGLEIIELACYRLLHKQPHLNSKKNE

SEQ ID NO: 3 - DNA, 2564 bp, Danio Rerio
  misc_feature (1586)..(1586): n is a, c, g, or t
gggaaacgatggctgccgtggtgaacatggggccagggccgcgtctgctggaggatttgctgcgggactcagagtgactgtgcgcgcctgcaggagtgcggaggagccgctgatttcctgagtttctcctgcggtcaaaccgcgctgaagctgaggatggtgtctcctcgtggtgtgtgtttcctcctcttcctcctgctgggcagtgtgttcggcagtgtgttcatgctgggtccgctgctgccgctgatgctgctgtctccgtcacgctaccgctggatcaccgaccgcatcgtggccacctggctgaccttacctgtggcgctgctggagctggtgttgggggtgaaggtggtcgtcacgggcgacgggtttatcccgggcgagcgcagcgtcatcatcatgaaccaccgcacacgcctggactggatgttcctgtggtgctgcttactgcgctacagctacctgcggcaggagaagatctgcctgaaggcggcgctcaagagtgtcccgggcttcggctgggcgatgcaggtcgcctccttcatcttcatccagcggcgctgggaggacgaccgcacacacatgagcaacatgctgcagtatttctgccgcatcagagagccggtgcagctgctgctgttccctgagggcaccgacctcaccgagaacactcgcgccaggagtgacgagtttgcggagaagaacgggcttcagaagtacgagtatgtgctgcacccgcgcaccaccggcttcaccttcatcgtggacacactgcgcggaggggataatctggatgcggttcatgacatcacagtggcgtatcctcagaatatcccgcagacggagcgccacctgctggcgggtgtgttcccgcgcgagatacacttccacgtgcagcgcttcacggtggcgtctgtgcccgcgggcgccgccggtctgcaggcgtggtgtcaggagcgctggcgggagaaggagcggcggctgcagcgcttctatgagacggtcccgcgccgcttcgatgcgcccgctgtgggtgtgtgtgtgcgtgagccgtgctgtcagagcgggcagtgtgtgtgtgtgccgcgctgtaagagcgaggggcgcgtgcgcatcatcctggtggcgtcgctgctctactggagcgtcttcatcaccgccgcctgcgccagcctctgcctctgcccccccgcacaattctacttcctgttcatggtcgtcttcttcctgtgtcagcagcggttcaccggcggcctggagctgatggagctggcctgccaccgatactggagccgccgcagcgccgacaagcaggagtgaagagtgtgtgtgtgtgtgtgtgtgtttgcaagcatgtgtgcatgacagtgtataggttattgaatgtgtgtataactgatgcataagtgtgtgtgtataaatgatgaatgagtgtagtgtgtgtgtgtgtgtgtgtagagctgctgctcctcctgctggctgagaggctgaagtgcagcacaacagacactcatataaacagaacagcagctcttcatcctccactcttcatcctcgcggtgacctttgactgtcagctgtanaggacagcggnggacactgatgtgacacacacacacacacacacacacacacacatttacttgatcaacacttgctctttaaagctgcagataatgaagagctcagaggaggtgtgtgtgtgtgtgtgtgcgtgtgcgtgtgtgtgtgtgtgtgtgtgtttggactcctctgagctgagtactgttctctgcagcgcaccccagcgcttcactcctcctcctcctcctcctcctcctcctctgtgtgtgtgtgcgtgtgtgtttgtgtgtgtgtgtgtgtgtgtgtgtgtgtctgtgtttgtgtgtgtgtgtgtgtgtgtgtgtgtgtgtgtgtgtgtgtgtgtgctgtggtggtgtgtcggctggtgtgtgtagtcctgcagtatactcgggtgttttagacgatcctcttttattatgggctgtgtctgtctgttattattagtgttttagttgaggatcacagccatagcggatgcgcacactgctcatggagaacagtgtggggaagacacacacacacacacacacacacttgtatcagtcatcagcacatgtaacaccagtacacagtgttctgcactgagagtgtttattctgtgtgtgttgtgttgatgagtgattgcagtgtgtgtgtgtgtgtgtgtttctgttttcctcctatgtgtgtgttgttcagctgtggtgttctgacctctctgggtcgcagtgggctggtttatactgtgtgttgaagaggaagatggagaagcacagaaacaccttcatcatcaggagatcagcctctgatcagtcaatcaataatcaatactctctctccacacctctgctgtgtcctctgcagtctgtgtgtgtccgtctgtcctcatctggctgcatttgtcatcttgagtatgtatttttctgatgtatagtatgatgaatgagtccagaataaacaggcctggcgttatccccaaaaaaaaaaaaaaaaaaaaa

SEQ ID NO: 4 - protein, 388 aa, Danio rerio
MVSPRGVCFLLFLLLGSVFGSVFMLGPLLPLMLLSPSRYRWTTDRIVATWLTLPVALLELVLGVKVVVTGDGFIPGERSVIIMNHRTRLDWMFLWCCLLRYSYLRQEKICLKAALKSVPGFGWAMQVASFIFIQRRWEDDRTHMSNMLQYFCRIREPVQLLLFPEGTDLTENTRARSDEFAEKNGLQKYEYVLHPRTTGFTFIVDTLRGGDNLDAVHDIAVAYPQNIPQTERHLLAGVFPREIHFHVQRFTVASVPAGAAGLQAWCQERWREKERRLQRFYETVPRRFDAPAVGVCVREPCCQSGQCVCVPRCKSEGRVRIILVASLLYWSVFITAACASLCLCPPAQFYFLFMVVFFLCQQRFTGGLELMELACHRYWSRRSADKQE

SEQ ID NO: 5 - DNA, 25 bp, Artificial Sequence
  Synthetic construct
aacacacaccacgaggagacaccat

SEQ ID NO: 6 - DNA, 24 bp, Artificial Sequence
  Synthetic Construct
taagctctgcgtaccacaggtaag

SEQ ID NO: 7 - DNA, 24 bp, Artificial Sequence
  Synthetic Construct
ctgaacacacacactgaccgaagc

SEQ ID NO: 8 - DNA, 24 bp, Artificial Sequence
  Synthetic Construct
gcagcgggcactgctggtggaagt

SEQ ID NO: 9 - DNA, 26 bp, Artificial Sequence
  Synthetic Construct
gatgtgtccgagaacacgcgtctgac

SEQ ID NO: 10 - DNA, 22 bp, Artificial Sequence
  Synthetic Construct
cctcagcctgacacacacacac

(19) United States
(12) Patent Application Publication
(10) Pub. No.: US 2010/0047246 A1
(43) Pub. Date: Feb. 25, 2010

(54) Methods and Compositions for Treating Vascular Disease

(75) Inventors: Jing-Wei Xiong, Andover, MA (US); Mark C. Fishman, Newton Center, MA (US)

Correspondence Address: CLARK & ELBING LLP, 101 FEDERAL STREET, BOSTON, MA, 02110, US

(73) Assignee: The General Hospital Corporation, Boston, MA (US)

(21) Appl. No.: 12/373,917

(22) PCT Filed: Jul. 17, 2007
(86) PCT No.: PCT/US07/16184

§ 371 Date: Jun. 30, 2009

Related U.S. Application Data
(60) Provisional application No. 60/831,944, filed on Jul. 18, 2006.

(51) Int. Cl. A61K 39/395; A61K 38/45; C12N 9/10; C12N 5/10; C12N 15/63; A61K 38/00; A61K 31/7088; C07H 21/04

(52) U.S. Cl. 424/139.1; 424/94.5; 435/193; 435/325; 435/320.1; 514/2; 514/44 R; 514/44 A; 536/23.2

(57) ABSTRACT

This invention relates to compositions and methods for use in treating or preventing vascular disease, as well as diseases and conditions associated with hematopoiesis and cellular proliferation.

### BACKGROUND OF THE INVENTION

This invention relates to methods and compositions for treating vascular disease.

Cardiovascular and ischemia-related diseases remain a major cause of mortality worldwide, despite recent advances in biomedical research and approaches to treatment. Complications of coronary, cerebral, and peripheral vascular disease, for example, account for approximately 50 percent of all mortality in the United States and are associated with an economic burden of over $300 billion dollars per year when hospitalization, therapeutic intervention, and lost productivity are considered. Additional conditions resulting in such personal, medical, and economic burdens include diabetes and cancer.

Vasculogenesis, angiogenesis, and hematopoiesis are developmentally related processes that, when disrupted, have been implicated in a variety of human diseases, including those noted above. Vasculogenesis involves the de novo differentiation of angioblasts into endothelial cells, which form blood vessels. Angiogenesis also involves endothelial cell growth, but results from the sprouting and growth of pre-existing blood vessels, rather than in the formation of completely new blood vessels. In adults, angiogenesis is required for the normal growth of postnatal tissues and accounts for transient phases of neo-vascularization during, for example, wound healing, the female reproductive cycle, and pregnancy in the placenta (Folkman, Nature Med. 1:27, 1995; Folkman and Shing, J. Biol. Chem. 267:10931, 1992; Risau, Nature 386:671, 1997). Dysfunctional angiogenesis has been implicated in pathological processes and conditions such as metastasis, solid tumor formation, rheumatoid arthritis, retinopathies, hemangiomas, and psoriasis (Edelberg and Reed, Front. Biosci. 8:s 1199, 2003; Folkman, Nature Med. 1:27, 1995; Hanahan and Folkman, Cell 86:353, 1996; Lakatta, Circulation 107:490, 2003). Hematopoiesis is the process by which blood cells (i.e., hematopoietic cells) are formed. Dysregulation of hematopoiesis can arise from disease (e.g., anemia, autoimmune disease, and congenital immune disorders) or as a side effect of the treatment of another disease (e.g., chemotherapy and radiation treatment of cancer).

The common developmental precursor to endothelial and hematopoietic cells is termed the “hemangioblast.” Characterization of hemangioblast differentiation is important for the development of treatment strategies for diseases related to vasculogenesis, angiogenesis, and hematopoiesis, such as those diseases noted above.

### SUMMARY OF THE INVENTION

The invention is based on our discovery of a gene, herein referred to as “lysocardiolipin acyltransferase,” “lycat,” “cloche,” or “clo,” as being necessary for hemangioblast and subsequent endothelial and hematopoietic cell differentiation.

Accordingly, the invention provides methods for identifying compounds that modulate the expression, stability, or activity of lycat. These methods can include the steps of: (a) incubating a lycat polypeptide or a lycat expression construct with a candidate compound; and (b) comparing the expression, stability, or activity of lycat in the presence of the candidate compound with the expression, stability, or activity of lycat in the absence of the candidate compound. These methods can be carried out in, for example, a cell-free mixture, a cell-based mixture, a recombinant cell, or an animal (e.g., a zebrafish, mouse, or human).

The invention also provides methods of inducing the production or development of hematopoietic or endothelial cells in a subject in need thereof, which involve administering to the subject an expression vector (e.g., an adeno-associated virus (AAV) vector) encoding lycat (e.g., human lycat). Further, the invention provides methods of inducing the production or development of hematopoietic or endothelial cells in a subject in need thereof, which involve administering to the subject a protein preparation of lycat (e.g., human lycat). In one example of such methods, the protein preparation includes a protein transduction domain-lycat fusion. Also provided by the invention are isolated nucleic acid molecules encoding lycat (e.g., a nucleic acid molecule including the sequence of SEQ ID NO:1 or SEQ ID NO:3). Further, the invention provides substantially purified proteins including the sequence of SEQ ID NO:2 or SEQ ID NO:4, and isolated nucleic acid molecules encoding these polypeptides. The nucleic acid molecules of the invention can, optionally, be operatively linked to expression control sequences. The invention also includes vectors including the nucleic acid molecules described herein, as well as cells (e.g., multipotent stem cells) including such vectors. The invention also includes use of such expression vectors in the preparation of medicaments for preventing or treating these diseases and conditions.

The invention further includes methods of differentiating stem cells into hemangioblasts, which involve introducing a vector such as that described above into a stem cell, as well as methods of producing hematopoietic or endothelial cells in subjects in need thereof, involving administration of such cells to the subjects. Diseases or conditions that can be treated or prevented according to the invention include ischemia-related conditions, as described further below, as well as conditions related to an insufficient number of hematopoietic cells (e.g., cancer, an autoimmune disorder, anemia, thalassemia, and a congenital immune deficiency disorder).

Also included in the invention are isolated nucleic acid molecules including siRNA that inhibits expression of lycat (e.g., nucleic acid molecules including the sequence of SEQ ID NO:5, 6, 7, or 8). Optionally, such nucleic acid molecules can be operatively linked to an expression control sequence and/or included within a vector. Such nucleic acid molecules can be used in methods of treating subjects for proliferative disorders. Further, the invention includes antibodies that specifically bind lycat, as described further elsewhere herein.

The invention also provides methods of determining whether a test subject (e.g., a mammal, such as a human) has or is at risk of developing a disease or condition related to lycat (e.g., a disease or condition of the vasculature, or a disease or condition related to hematopoiesis or hyperproliferation). These methods can involve analyzing a nucleic acid molecule of a sample from the test subject to determine whether the test subject has a mutation in a gene encoding lycat. Detection of the presence of a mutation indicates that the test subject has or is at risk of developing a disease or condition related to lycat.

The invention further includes methods of inducing the production or development of hematopoietic or endothelial cells in a subject in need thereof, which involve administering to the subject a compound that activates expression of lycat, stabilizes lycat message or protein, or increases lycat activity. Also, the invention provides methods of treating a subject for a proliferative disorder, involving administering to the patient a compound that decreases expression of lycat, destabilizes lycat message or protein, or decreases lycat activity.

By “lysocardiolipin acyltransferase,” “lycat,” “cloche,” and “clo” (for convenience, the term “lycat” is generally used herein) is meant any polypeptide having the activity of full-length lycat protein (e.g., human or zebrafish lycat). Examples of such polypeptides include full-length lycat, such as human lycat (e.g., SEQ ID NOs:1 and 2 (Hclo)), zebrafish lycat (e.g., SEQ ID NOs:3 and 4), and xenologues thereof (e.g., murine lycate (Mclo))

The terms “lysocardiolipin acyltransferase,” “lycat,” “cloche,” and “clo” also are used herein to refer to lycat fragments, which may be, e.g., functional, antigenic, and/or immunogenic. Examples of such fragments include those that consist of, consist essentially of, or include the lycat acyltransferase domain. Further, these terms also encompass lycat polypeptides or fragments including additional terminal amino acids, e.g., an amino terminal methionine.

The terms “lysocardiolipin acyltransferase,” “lycat,” “cloche,” and “clo” also are used herein to refer to fusions of full-length lycat, or fragments thereof, with one or more other polypeptides. Examples of such fusions include glutathione-S-transferase (GST)-lycat, hemagglutinin (HA)-tagged lycat, and Flag-tagged lycat. These additional polypeptides may be linked to the N-terminus and/or C-terminus of lycat.

The terms “lysocardiolipin acyltransferase,” “lycat,” “cloche,” and “clo” are also used herein to refer to proteins or peptides having at least 45%, e.g., at least 60%, 75%, or 90% (e.g., 91%, 92%, 93%, 94%, 95%, 96%, 97%, 98%, or 99%) amino acid sequence identity to the sequence of a human (SEQ ID NO:2) or a zebrafish (SEQ ID NO:4) lycat polypeptide. Polypeptide products from any splice variants of lycat gene sequences and lycat genes containing mutations are also included in this definition.

The terms “lysocardiolipin acyltransferase,” “lycat,” “cloche,” and “clo” also herein refer to polypeptides having sequences that include conservative substitutions of amino acid residues in lycat. The term “conservative substitution” refers to replacement of an amino acid residue by a chemically similar residue, e.g., a hydrophobic residue for a different hydrophobic residue, a charged residue for a different charged residue, etc. Examples of conservative substitutions for non-polar R groups are alanine, valine, leucine, isoleucine, proline, methionine, phenylalanine, and tryptophan. Examples of substitutions for polar, but uncharged R groups are glycine, serine, threonine, cysteine, asparagine, and glutamine. Examples of substitutions for negatively charged R groups are aspartic acid and glutamic acid. Examples of substitutions for positively charged R groups are lysine, arginine, and histidine. In addition to the substitutions noted above, such polypeptides can also include substitutions with non-natural amino acids.

By “polypeptide,” “polypeptide fragment,” or “peptide” is meant a chain of two or more (e.g., 10, 15, 20, 30, 50, 100, or 200, or more) amino acids, regardless of any post-translational modification (e.g., glycosylation or phosphorylation), constituting all or part of a naturally or non-naturally occurring polypeptide, fragment, or peptide. By “post-translational modification” is meant any change to a polypeptide or polypeptide fragment made during or after synthesis. Post-translational modifications can be produced naturally (such as during synthesis within a cell) or generated artificially (such as by recombinant or chemical means). A “protein” can be made up of one or more polypeptides.

By a “lysocardiolipin acyltransferase,” “lycat,” “cloche,” and “clo” nucleic acid molecule is meant a nucleic acid molecule, such as a genomic DNA, cDNA, or RNA (e.g., mRNA) molecule, that encodes a lycat protein (e.g., a human (encoded by SEQ ID NO:1) or a zebrafish (encoded by SEQ ID NO:3) lycat protein), lycat polypeptide, or fragment thereof, as referred to above.

A mutation in a lycat nucleic acid molecule can be characterized, for example, by the insertion of a premature stop codon anywhere in the lycat gene, or by a mutation in a splice donor site, which leads to aberrant transcript production (e.g., transcripts with premature stop codons). In addition to cloche and similar mutations (see below), the invention includes any mutation that results in aberrant lycat gene expression, message stability, protein production, and/or protein function, including, only as examples, null mutations and mutations causing truncations.

The term “identity” is used herein to describe the relationship of the sequence of a particular nucleic acid molecule or polypeptide (or a fragment thereof) to the sequence of a reference molecule of the same type (or a fragment thereof). For example, if a nucleic acid or amino acid molecule has the same nucleotide or amino acid residue at a given position, as compared to a reference molecule to which it is aligned, there is said to be “identity” at that position. The level of sequence identity of a nucleic acid molecule or a polypeptide to a reference molecule is typically measured using sequence analysis software with the default parameters specified therein, such as the introduction of gaps to achieve an optimal alignment (e.g., Sequence Analysis Software Package of the Genetics Computer Group, University of Wisconsin Biotechnology Center, 1710 University Avenue, Madison, Wis. 53705, BLAST, or PILEUP/PRETTYBOX programs). These software programs match identical or similar sequences by assigning degrees of identity to various substitutions, deletions, or other modifications.

The sequence of a nucleic acid molecule or polypeptide is said to be “substantially identical” to that of a reference molecule if it exhibits, over its entire length, at least 51%, e.g., at least 55%, 60%, 65%, 75%, 85%, 90%, or 95% (e.g., at least 91%, 92%, 93%, 94%, 95%, 96%, 97%, 98%, or 99%) identity to the sequence of the reference molecule. For nucleic acid molecules, the length of comparison sequences may be, for example, at least 20, 30, 40, 50, 60, 75, or 100 nucleotides. For polypeptides, the length of comparison sequences may be, for example, at least 16, 20, 25, or 35 amino acids. Of course, the length of comparison can be any length, up to and including full length.

A nucleic acid molecule or polypeptide of the invention is “analyzed” or subject to “analysis” if a test procedure is carried out on it that allows the determination of its biological activity or whether it is wild type or mutated. For example, one can analyze the lycat genes of an animal (e.g., a human or a zebrafish) by amplifying genomic DNA of the animal using the polymerase chain reaction, and then determining whether the amplified DNA contains a mutation, for example, the cloche mutation, by, e.g., nucleotide sequence or restriction fragment analysis.

By “probe” or “primer” is meant a single-stranded DNA or RNA molecule of defined sequence that can base pair to a second DNA or RNA molecule that contains a complementary sequence or partially complementary (a “target”). The stability of the resulting hybrid depends upon the extent of the base pairing that occurs. This stability is affected by parameters such as the degree of complementarity between the probe and target molecule, and the degree of stringency of the hybridization conditions. The degree of hybridization stringency is affected by parameters such as the temperature, salt concentration, and concentration of organic molecules, such as formamide, and is determined by methods that are well known to those skilled in the art. Probes or primers specific for lycat nucleic acid molecules may have, e.g., greater than 45, 55-75, 75-85, 85-99, or 100% sequence identity to the sequences of human (SEQ ID NO:1) or zebrafish (SEQ ID NO:3) lycat genes. Further, such probes or primers may, optionally, include modified nucleotides, e.g., nucleotides that enhance the stability of the molecules.

Probes can be detectably labeled, either radioactively or non-radioactively, by methods that are well known to those skilled in the art. Probes can be used for methods involving nucleic acid hybridization, such as nucleic acid sequencing, nucleic acid amplification by the polymerase chain reaction, single stranded conformational polymorphism (SSCP) analysis, restriction fragment polymorphism (RFLP) analysis, Southern hybridization, northern hybridization, in situ hybridization, electrophoretic mobility shift assay (EMSA), and other methods that are well known to those skilled in the art.

A molecule, e.g., an oligonucleotide probe or primer, a gene or fragment thereof, a cDNA molecule, a polypeptide, or an antibody, can be said to be “detectably-labeled” if it is marked in such a way that its presence can be directly identified in a sample. Methods for detectably labeling molecules are well known in the art and include, without limitation, radioactive labeling (e.g., with an isotope, such as ³²P or ³⁵S) and nonradioactive labeling (e.g., with a fluorescent label, such as fluorescein).

By a “substantially pure” polypeptide is meant a polypeptide (or a fragment thereof) that has been separated from proteins and organic molecules that naturally accompany it. Typically, a polypeptide is substantially pure when it is at least 60%, by weight, free from the proteins and naturally occurring organic molecules with which it is naturally associated. For example, the polypeptide can be a lycat polypeptide that is at least 75%, 90%, or 99%, by weight, pure. A substantially pure lycat polypeptide can be obtained, for example, by extraction from a natural source, by expression of a recombinant nucleic acid molecule encoding a lycat polypeptide, or by chemical synthesis. Purity can be measured by any appropriate method, e.g., by column chromatography, polyacrylamide gel electrophoresis, or HPLC analysis.

A polypeptide is substantially free of naturally associated components when it is separated from those proteins and organic molecules that accompany it in its natural state. Thus, a protein that is chemically synthesized or produced in a cellular system that is different from the cell in which it is naturally produced is substantially free from its naturally associated components. Accordingly, substantially pure polypeptides not only include those that are derived from eukaryotic organisms, but also those synthesized in E. coli, other prokaryotes, or in other such systems.

By “isolated” nucleic acid molecule is meant a nucleic acid molecule that is removed from the environment in which it naturally occurs. For example, a nucleic acid molecule present in the genome of cell in which it naturally occurs is not isolated, but the same molecule, separated from the remaining part of the genome, as a result of, e.g., a cloning event (amplification), is “isolated.” Typically, an isolated nucleic acid molecule is free from nucleic acid regions (coding or non-coding regions) with which it is immediately contiguous, at the 5′ or 3′ ends, in the naturally occurring genome. Such isolated nucleic acid molecules can be part of a vector or a composition and still be isolated, as such a vector or composition is not part of its natural environment.

An antibody is said to “specifically bind” to a polypeptide, peptide, or fragment if it recognizes and binds to the polypeptide (e.g., a lycat polypeptide), but does not substantially recognize and bind to other molecules (e.g., non-lycat-related polypeptides) in a sample, e.g., a biological sample that includes the polypeptide.

By “high stringency” conditions is meant conditions that allow hybridization comparable with the hybridization that occurs using a DNA probe of at least 50, e.g., 100, 200, 350, or 500, nucleotides in length, in a buffer containing 0.5 M NaHPO₄, pH 7.2, 7% SDS, 1 mM EDTA, and 1% BSA (fraction V), at a temperature of 65° C., or a buffer containing 48% formamide, 4.8×SSC, 0.2 M Tris-HCl, pH 7.6, 1×Denhardt's solution, 10% dextran sulfate, and 0.1% SDS, at a temperature of 42° C. (These are typical conditions for high stringency northern or Southern hybridizations.) High stringency hybridization is also relied upon for the success of numerous techniques routinely performed by molecular biologists, such as high stringency PCR, DNA sequencing, single strand conformational polymorphism analysis, and in situ hybridization. In contrast to northern and Southern hybridizations, these techniques are usually performed with relatively short probes (e.g., usually 16 nucleotides or longer for PCR or sequencing, and 40 nucleotides or longer for in situ hybridization). The high stringency conditions used in these techniques are well known to those skilled in the art of molecular biology, and examples of them can be found, for example, in Ausubel et al., Current Protocols in Molecular Biology, John Wiley & Sons, New York, N.Y., 1998, which is incorporated herein by reference.

By “sample” is meant a tissue biopsy, amniotic fluid, cell, blood, serum, urine, stool, or other specimen obtained from a patient or a test subject. The sample can be analyzed to detect a mutation in a lycat gene or expression levels of a lycat gene by methods that are known in the art. For example, methods such as sequencing, single-strand conformational polymorphism (SSCP) analysis, or restriction fragment length polymorphism (RFLP) analysis of PCR products derived from a patient sample can be used to detect a mutation in a lycat gene; ELISA and other immunoassays can be used to measure levels of a lycat polypeptide; and PCR or RT/PCR can be used to measure the level of a lycat nucleic acid molecule.

The terms “lysocardiolipin acyltransferase-related disease,” “lycat-related disease,” “cloche-related disease,” “clo-related disease,” “lysocardiolipin acyltransferase-related condition,” “lycat-related condition,” “cloche-related condition,” or “clo-related condition” include diseases or conditions that results from inappropriately high or low expression of a lycat gene, or a mutation in a lycat gene (including control sequences, such as promoters) that alters the biological activity of a lycat nucleic acid molecule or polypeptide. Lycat-related diseases and conditions can arise in any tissue in which lycat is expressed during prenatal or post-natal life. Lycat-related diseases and conditions can include diseases or conditions of the vascular and hematopoietic systems, as is described further below.

The invention provides several advantages. For example, increasing lycat levels and/or activity can be useful in the treatment of diseases in which benefit would be obtained by promoting vasculogenesis, angiogenesis, and/or hematopoiesis. Thus, as discussed further below, approaches according to the present invention can be used in the treatment of, for example, ischemia-related, vascular, anemic, autoimmune, and congenital immune disorders. Further, decreasing lycat levels and/or activity can be useful for treating proliferative disorders including, e.g., cancer. Also, the screening methods of the invention can be used to identify compounds that modulate lycat expression or activity, which can be used in the therapeutic methods described herein. The invention thus provides substantial benefits to a large number of patients suffering from the diseases and conditions described herein, and thus contribute to alleviating the substantial personal, medical, and economic burdens of these diseases and conditions.

Other features and advantages of the invention will be apparent from the following detailed description, the drawings, and the claims.

### BRIEF DESCRIPTION OF THE DRAWINGS

FIG. 1. The do mutant lacks the endocardium and most vascular and hematopoietic cells labeled with tissue-specific GFP in zebrafish embryos. 1a and 1b, EGFP expression under control of the Flk1 promoter in vessels and the intermediate cell mass (ICM) of wild-type embryos (a), and very few EGFP-positive cells in homozygous do embryos (b) at 24 hpf. 1c and 1d, EGFP expression under control of the GATA1 promoter in the ICM in wild-type embryos (c), and very few EGFP-positive cells in clo embryos (d) at 24 hpf. 1e and 1f, Tg[flk1:GFP] expression in the endocardium of wild-type embryos (e), but not the heart (arrow points) of clo embryos (f) at 48 hpf. 1g and 1h, similar EGFP expression under control of the heart-specific myosin light chain 2 (mlc2) promoter in the myocardium of wild-type (g) and clo (h) embryos at 48 hpf. 1i and 1j, transgenic wild-type (i) and clo (j) Tg[Flk1:gfp] embryos, showing very few GFP+ cells in the ICM of the clo embryo at 24 hpf.

FIG. 2. A zebrafish lycat homologue was isolated from the clo^{m39} deletion interval. a, A genetic and physical map of the cloche locus. Cloche was mapped on the telomere of chromosome 13 close to the microsatellite markers Z17223, Z22194, and Z10362 (Knapik et al., Nature Genet. 18:338, 1998) using bulked segregant analysis. Fine mapping was done with 4173 clo^{fv087b} mutants. The clo^{fv087b} interval was defined between Z12051 and a microsatellite marker D derived from BAC109E22 (Incyte-Zebrafish BAC by Dr. Peter de Jong). Using a shotgun sequencing approach, we have obtained 5× coverage in the sequences of YAC2 (YAC2=35K05 from the BCH-YAC library), PAC1 (PAC1=60K08 from the BAC library by Dr. Chris Amemiya), and BAC4 (109E22). We assembled several contigs, and found a single contig 35 kb in length that covers the critical clo interval, and derived microsatellite markers from CA repeats in the assembled sequences. Bioinformatic tools including Genscan and BLAST were used to identify exons, introns, and genes in this contig. Six zebrafish genes including Peas (Accession number NM_{—}057161), PACS1a (NM_{—}018026), CGI-127 (AF172940), LYCAT/ALCAT1 (NM_{—}182551), LBH (NM_{—}030915), and hole (BC004221) were found (FIG. 2a). The clo^{m39} allele is a spontaneous mutation (Stainier et al., Development 121:3141, 1995), and was subsequently discovered to have a deletion during the course of cloche mapping (FIG. 2a). The clo^{m39} deletion spans less than 0.2 cM between Z1826 and the T7 end of BAC4. b, The zebrafish lycat cDNA isolated by positional cloning is identical to the full-length cDNA clone deposited in the GeneBank (BC066444) that contains 2452 nucleotides and encodes a 388 amino acid protein (FIG. 2b). Lycat (SEQ ID NO:4) has a conserved acyltransferase domain (gray), three putative transmembrane domains (underlined), and a putative endoplasmic reticulum retention signal (hold and underlined).

FIG. 3. Antisense lycat morpholinos mimic the cloche mutant phenotype and lycat mRNA rescues morpholino mediated knockdown. Three lycat morpholinos were designed to target the zebrafish lycat exon 2 splice donor site (d322), the exon 3 splice donor site (d522), and the translational start site (ATG). a-d, 0.3 mM d522-injected morphants at 36 hpf (c) and at 48 hpf (d) as well as the 0.5 mM a345 control morpholino injected embryos at 36 hpf (a) and at 48 hpf (b). e-g, strong flk1:GFP signals in vessels of a345-injected control embryo (e), very little flk1:GFP signals in 0.3 mM d522-injected embryo (f), and rescued flk1:GFP signals in the embryo coinjected with 0.3 mM d522 and 300 ng/ul lycat RNA (g) at 24 hpf. h-j, strong gata1:GFP signal in the blood islands of a345-injected control embryo (h), very little gata1:GFP signal in 0.3 mM d522-injected embryo (i), and rescued gata1:GFP signal in the embryo coinjected with 0.3 mM d522 and 300 ng/ul lycat RNA (j). k-n, the scl expression in the ICM in control-injected embryo (k), decreased scl in embryos injected with 0.3 mM d522 (i), 0.4 mM d522 (j), and 0.5 mM d522(n) at 20 hpf by RNA in situ analysis.

FIG. 4. Zebrafish lycat and mouse lycat RNA rescues the cloche phenotype and can increase scl⁺ cells in 24 hpf zebrafish embryos. Strong gata1:GFP expression in the blood island (arrow) and spinal chord gata1-GFP expression (arrowhead) in wild type embryos (a), very little gata1:GFP in cloche mutants injected with the zebrafish antisense lycat RNA (arrow) (b), rescued gata1-gfp in cloche mutant with zebrafish sense lycat RNA (c) and with mouse sense lycat RNA (d) at 20 hpf. Scl⁺ cells are detected by RNA in situ hybridization in embryos injected with control RNA (e), and lycat mRNA injected embryos (f). The arrow indicates ectopic scl-expressing cells in the anterior part of embryos.

FIG. 5. Zygotic lycat RNA is expressed in the ICM, circulating blood, and the heart. a, maternal and zygotic lycat mRNA expression in embryos from 0 to 36 hpf by RT-PCR. β-actin was used as an internal RNA control. b-d, using a lycat:Gal4-VP16 and UAS-GFP system, GFP expression driven by a 1742 bp lycat promoter is detected in the ICM and blood islands at 22 hpf (c). 3b is a bright-field (BF) picture of 3c. 3d, strong lycat:GFP signal in the heart of transgenic embryo at 72 hpf (bright field and fluorescence images superimposed using Metaview software).

FIG. 6. Expression constructs of mouse clo (Mclo). Transgenic expression of Mclo is directed by the mouse Flk1 promoter (Flk1P) and enhancer (Flk1E), or β-actin promoter (β-actin) and CMV-IE enhancer. A PGKneo cassette was inserted for G418 selection in transfected ES cells to form the final constructs Flk1:Mclo-iresEGFP;PGKneo and β-actin:Mclo-iresEGFP;PGKneo. Flk1P, Flk1 promoter; pA, SV40 polyadenylation sequence; Flk1E, Flk1 enhancer; βActin, β-actin promoter and CMV-IE enhancer.

FIG. 7. Mouse lycat is enriched in the Flk1⁺/Scl⁺ hemangioblast, and its overexpression in mouse ES cells can increase Flk1⁺ endothelial and CD45⁺ pan-hematopoietic cells. a, Mouse lycat is expressed in the Flk1⁺ mesoderm and hemangioblasts, but not in Flk1⁻ cells. The Scl:hCD4 ES cells were induced to form embryoid bodies in a suspension culture (Chung et al., Development 129:5511, 2002). Day-4 EBs were dissociated and sorted using anti-Flk1 and anti-hCD4 (i.e., Scl). RNA was extracted, first-strand cDNA was synthesized, and semi-quantitative PCR was carried out for amplifying lycat or β-actin (internal control). b, mouse lycat RNA expression is increased in transgenic clones overexpressing mouse lycat driven by the Flk1 promoter (Wu et al., Mol. Cell. Biol. 23:5680, 2003). R1 ES cells, control transgenic R1 ES clones without mouse lycat, and transgenic R1 ES clones (RFC5, 13, and 22) overexpressing the lycat transgene were differentiated into day-4 EBs. Semi-quantitative RT-PCR analysis of lycat expression was carried out in day-4 EBs. Mouse lycat has very low expression in R1 and control (Contr) transgenic R1 ES-derived EBs, but has strong lycat RNA expression in transgenic lycat ES-derived EBs (RFC5, 13, and 22). R1, R1 EBs; Contr, R1 ES cell transfected with Flk1:GFP DNA without mouse lycat. β-actin was used as an internal control for RT-PCR. c, Over-expression of lycat driven by the mouse flk1 promoter causes a two-fold increase of Flk1⁺ cells (d-g), and a five-fold increase of CD45⁺ hematopoietic cells (I-1) in day-4 embryoid bodies derived from stable transgenic lycat ES cell clones RFC1 (d, i), RFC5 (e, j), RFC7 (f, k), and RFC22 (g, l), as compared with that from the control transgenic ES cell clone without the lycat transgene (c, h). This represents one of three independent experiments.

FIG. 8. Lycat modulates VEGF protein activities in zebrafish. Numbers on the Y-axis represent numbers of treated embryos with or without clo phenotypes. The light gray and dark gray columns represent numbers of do and non-clo embryos, respectively. VEGF MO stands for anti-sense VEGF morpholino, lycat MO for the splicing lycat morpholino, and KDRi for 6 μM A676475 of KDR tyrosine kinase inhibitor (Calbiochem, San Diego, Calif.). Either VEGF MO or KDRi with low dose lycat MO could lead to generating a do phenotype (light gray) in the treated embryos (dark gray).

FIG. 9. Mclo mRNA and protein expression. a, RT-PCR amplification of a 1197 bp fragment of Mclo cDNA was carried out from mouse embryos (E7.5, E9.5, E11, E15, and E17) and adult organs including liver (Li), brain (Br), heart (Hr), lung (Lu), prostate (Pr), salivary gland (Sg), smooth muscle (Sm), spleen (Sp), stomach (St), testis (Te), thymus (Th), and uterus (Ut). b, hematopoietic cells were sorted from BM by FACS with anti-Sca1 and C-Kit, including Sca1⁺/C-Kit⁻ (S⁺C⁻), Sca1⁺ (S⁺), C-Kit⁺ (C⁺), and Sca1⁺/C-Kit⁺ (S⁺C⁺) populations. Mclo mRNA was enriched in C-Kit⁺ and Sca1⁺/C-Kit⁺, but not in Sca1⁺/C-Kit⁻ and Sca1⁺ cells using RT-PCR. β-actin was used as an internal control for RNA input. c, Mclo protein is expressed in the adult heart (Hrt) and not in skeletal muscle (SM) as shown by Western Blotting analysis with anti-Mclo. Mclo protein is about 42 kD as predicted (arrow). This is consistent with Mclo mRNA expression in the heart, but not in skeletal muscle as shown by others (Cao et al., J. Biol. Chem. 279:31727, 2004). d, hematopoietic lineages were sorted from BM by FACS, including Ter119⁺ (erythrocytes), Gr-1⁺ (myeloid/myeloid), Flk1⁺ (endothelial cells), B220⁺ (B cells), and WBH (whole bone marrow) as a control. Mclo mRNA was enriched in lymphocytes (B220⁺), but has very little in Ter119⁺, Gr-1⁺, and Flk1⁺ cells using RT-PCR. β-actin was used as an internal control for RNA input.

FIG. 10. Murine lycat is an ER-associated acyltransferase. a-c, COS7 cells were transiently transfected with pRY1 lycat-EGFP, in which the C-terminal of the lycat protein was fused in frame with N-terminal of EGFP (a-c), or with pRY2 EGFP-lycat, in which the N-terminal of lycat was fused in frame with the C-terminal of EGFP (d-f). EGFP signal of the fusion protein (a,d) and immunostaining with the ER marker PDI (b,e) of the same cell, as well as the superimposed of EGFP and PDI staining (c,f) are shown. EGFP-lycat fusion proteins co-localized well with the ER marker PDI (d-f), but lycat lost its ER localization (a-c) if EGFP was fused to block the C-terminal ER retention signal in lycat.

FIG. 11. Whole mount RNA in situ analysis of Egf17 and Mclo (mouse lycat) genes in mouse embryos. Embryos at E7.5 (a), E8.5 (c), and E9.5(d) were fixed and hybridized with Egf17 anti-sense probes; and an embryo at E8.5 (b) was fixed and hybridized with Egf17 sense probes as a control. Embryos at E8.5 (e) and E 9.5(f) were fixed hybridized with anti-sense Mclo probes. Egf17 has expression in the blood island (arrow head, 10a), dorsal aorta (arrow, 10c), and whole vasculature and the endocardium (10d). Mclo has expression in the aorta (arrow, 10e) and the heart (10f). Embryos have very little staining after probing with sense probes of Egf17 (b) and Mclo.

FIG. 12. Overexpression of Mclo could increase the Flk1⁺/Scl⁺ hemangioblasts in ES. 2a, Mclo is expressed in the Flk1+mesoderm and hemangioblasts but not in Flk1⁻ cells. The hCD4-Scl ES cells (Chung et al., Development 129:5511, 2002) were induced to form embryoid bodies in a suspension culture (Xiong et al., Dev. Dyn. 212:181, 1998). Day-4 EBs were dissociated and sorted using anti-Flk1 and Scl. RNA was extracted, first-strand cDNA was synthesized, and semi-quantitative PCR was carried out for amplifying Mclo or β-actin as an internal control. The Mclo mRNA was enriched in the Flk1⁺/Scl⁻ and Flk1⁺/Scl⁺ cells, but not in Flk1⁻/Scl⁺ and Flk1⁻/Scl⁻ cells. 1b-f, over-expression of Mclo could increase 2-3 fold over Flk1⁺/Scl⁺ hemangioblasts using FACS analysis with anti-Flk1 and -hCD4. 1b, negative; 1c, control hCD4-Scl ES; 1d-1f, three independent hCD4-SCL ES clones over-expression of Mclo driven by the Flk1 promoter.

FIG. 13. Overexpression of Mclo driven by the flk1 promoter increased the putative definitive HSCs in day-5 EBs. a-d, over-expression of Mclo could increase about 7 fold over CD41⁺ (c,d) and 2 fold over CD41⁺/C-Kit⁺ (g,h) cells using FACS analysis with anti-CD41 and -C-Kit. (a,e), negative control; (b,f), control ES clone without Mclo. This represents one of two independent experiments.

FIG. 14. The mouse Mclo transgene increases the formation of blast colony forming cells (BL-CFCs) in mouse ES cell-derived EBs. Day-4 EBs were dissociated and replated methylcellulose medium with growth factors for four days. before counting (Faloon et al., Development 127:1931, 2000). 20,000 cells were used for BL-CFC formation. Triplicates were done for each ES clone. The numbers of BL-CFCs were scored under a microscope and are expressed as BL-CFCs/20,000 cells. Control, control transgenic R1 ES clones without mouse Mclo; RFC2, 5 and 22 are three independent R1 ES clones over-expressing mouse Mclo driven by the mouse Flk1 promoter. This represents one of two independent experiments. The right picture shows a typical cluster of BL-CFCs.

FIG. 15. Mclo (mouse lycat) RNAi inhibit the Flk1+ and Scl+hemangioblast formation in embryoid bodies. Two Mclo RNAi, called RNAi78 and RNAi83, were cloned into the pSilencer 1.0 vector so that the RNAi is under control of the U6 promoter. The Mclo RNAi construct DNA was transfected with a PGK-neo construct DNA into hCD4_Scl ES cells. Embryoid bodies were differentiated from the hCD4_Scl ES cells (the control), RNAi78 and RNAi83 G418-resistant cells. The Flk1+, Scl/hCD4+, and PECAM1+ cells were determined using FACS with Flk1, hCD4, and PECAM1 antibodies, respectively.

### DETAILED DESCRIPTION

We have identified a gene, herein referred to as “lysocardiolipin acyltransferase,” “lycat,” “cloche,” or “clo,” as being necessary for hemangioblast and subsequent endothelial and hematopoietic cell differentiation. Increasing lycat levels and/or activities in patients can thus be used in approaches to treat diseases or conditions in which benefit would be obtained by stimulating blood vessel and/or hematopoietic cell growth or differentiation. As is discussed further below, lycat levels and/or activity can be increased by administration of lycat itself, genes encoding lycat (e.g., genes in vectors such as viral vectors), cells expressing lycat, or compounds that, for example, activate lycat expression, stabilize lycat, and/or increase lycat activity. Decreasing lycat levels can be used in approaches to treating diseases or conditions that would benefit from decreasing or blocking blood vessel or hematopoietic cell growth or development. This can be achieved, for example, by administration of siRNA directed against lycat or compounds that decrease lycat expression, stability, and/or activity. Further, detecting mutations in lycat genes can be used in the diagnosis of, for example, diseases and conditions associated with aberrant hemangioblast development or differentiation.

Diseases and conditions that can be treated or diagnosed according to the invention are described below, followed by details as to therapeutic and diagnostic methods that can be used with respect to these diseases and conditions, according to the invention, as well as methods for identifying agents that can be used in the therapeutic and diagnostic methods. Also provided below are the details of experimental results supporting the invention.

### Diseases and Conditions

As is noted above, increasing lycat levels, stability, and/or activity in patients can be used in approaches to treating diseases or conditions in which benefit would be obtained by stimulating blood vessel and/or hematopoietic cell growth or differentiation. Examples of diseases or conditions benefiting from stimulation of blood vessel growth include ischemia-related diseases or conditions, which are characterized by insufficient blood supply to a tissue or organ and which can result in damage or dysfunction of the tissue or organ. Ischemia that can be treated according to the invention can occur in any tissue or organ including, for example, heart, brain, intestine, liver, kidney, muscle, and eye, and can be due to any number of causes including, for example, vascular occlusion (caused by, e.g., plaque formation or thrombosis) or other injury. Thus, specific examples of ischemia-related diseases or conditions include heart attack, myocardial ischemia, stroke, diabetic ischemia, critical limb ischemia, and intestinal ischemia. Revascularization is typically a critical component of wound healing and, thus, the approaches of the invention can be used to promote wound healing in any of a number of contexts including, e.g., accident victims. Further, the methods of the invention can be used to promote vascularization of transplanted tissues or organs.

Increasing hematopoiesis, according to the invention can be used in the treatment of many different diseases and conditions. For example, the methods of the invention can be used to treat patients suffering from any of a number of different types of anemia (i.e., red blood cell or erythrocyte deficiency) including, for example, anemia due to kidney disease, hemolytic anemia (e.g., autoimmune hemolytic anemia, sickle cell anemia, thalassemia (e.g., thalassemia major or thalassemia minor), and glucose-6-phosphate dehydrogenase deficiency), and aplastic anemia (e.g., anemia due to iron, vitamin B12 (pernicious anemia), or folate deficiency; viral infection; or exposure to toxic agents).

In addition to treating diseases or conditions that benefit from promoting the growth of myeloid cells, such as erythrocytes (as discussed above), the methods of the invention involving increasing hematopoiesis can also be used to treat diseases or conditions that benefit from promoting the growth of other myeloid cells (e.g., neutrophils, monocytes, macrophages, eosinophils, megakaryocytes, mast cells, and basophils), as well as lymphoid cells (e.g., plasma cells, memory B cells, cytotoxic T lymphocytes, memory T lymphocytes, Th1 lymphocytes, Th2 lymphocytes, and precursors thereof). Thus, the methods of the invention can be used to treat patients suffering from diseases and conditions characterized by leukopenia, neutropenia, and thrombocytopenia.

Cancer patients in whom therapeutic regimens, such as chemotherapy and radiation, have resulted in decreased production of blood cells are a specific example of a group of patients that can benefit from treatment according to the invention. These patients may suffer from anemia, as well as leucopenia and neutropenia. As other specific examples, the methods of the invention can be used to treat accident victims, surgical patients, and patients with clotting disorders who have suffered or are at risk of suffering substantial blood loss.

In addition to methods of increasing vascular growth and/or hematopoiesis, the invention also provides methods of decreasing these processes, which can be used in the treatment and prevention of many diseases and conditions including, for example, hyperproliferative diseases such as cancer. As an example, the growth of solid tumors can be prevented or inhibited, according to the invention, as continued growth of such tumors may require vascularization of the tumor. In a related example, cancer metastasis can be treated or prevented according to the invention, as metastases require vascularization to be established and grow. In addition to solid tumors and metastases, the methods of the invention can be used in the treatment of cancers of the blood including, for example, acute myelogenous leukemia (AML) (e.g., undifferentiated AML, myeloblastic leukemia (M1), myeloblastic leukemia (M2), promyelocytic leukemia (M3 or M3 variant), myelomonocytic leukemia (M4 or M4 variant with eosinophilia (M4E)), monocytic leukemia (M5), erythroleukemia (M6), erythroleukemia (M6)), Chronic Myelogenous Leukemia (CML), Acute Lymphocytic Leukemia (ALL), Chronic Lymphocytic Leukemia (CLL), and hairy cell leukemia.

Further, the methods of the invention can be used to treat diseases or conditions characterized by non-cancerous hyperproliferation or dysregulation of immune system cells including, e.g., autoimmune diseases, such as rheumatoid arthritis, lupus (systemic lupus erythematosus), psoriasis, scleroderma, Sjogren's syndrome, Goodpasture's syndrome, Wegener's granulomatosis, polymyalgia rheumatica, temporal arteritis, type I diabetes mellitus, Hashimoto's thyroiditis, Grave's disease, celiac disease, ulcerative colitis, Crohn's disease, multiple sclerosis, Guillain-Barre syndrome, Addison's disease, primary biliary sclerosis, sclerosing cholangitis, autoimmune hepatitis, Raynaud's phenomenon, myasthenia grava, dermatomyositis, and Reiter's syndrome.

The methods of the invention can be used as sole treatment modalities or can be used in conjunction with other techniques. For example, in the case of myocardial ischemia, the methods of the invention can be used in combination with revascularization techniques such as balloon angioplasty, stenting, and/or coronary artery bypass grafting. As another example, approaches to increasing hematopoiesis, according to the invention, can be carried out in combination with approaches such as treatment with hematopoietic growth factors (e.g., erythropoietin, CSFs (e.g., GM-CSF, M-CSF, and G-CSF), interleukins (e.g., any of ILs 4-9), and bone marrow transplantation.

Increasing lycat levels and/or activity can be achieved through methods including cell-based therapy, gene therapy, protein therapy, and pharmaceutical (agonist) therapy, while decreasing lycat levels and/or activity can be achieved through methods including RNAi, antibody, and pharmaceutical (antagonist) therapy. Examples of these types of therapeutic approaches, which can be used in the invention, are provided below.

### Cell-Based Therapies

As is discussed above, hemangioblasts are a common precursor for cells of both endothelial and hematopoietic lineages. Thus, therapy based on the administration of hemangioblasts can be used to treat diseases or conditions that can benefit by increased vascular growth and/or hematopoiesis, as described herein. Also as discussed above, the present invention is based on the discovery that lycat is required for hemangioblast formation and differentiation. Based on this discovery, it is now known that activation of lycat in hemangioblast precursors (i.e., stem cells) can be used, according to the invention, in the generation of hemangioblasts for use in therapeutic methods.

Stem cells that can be used in the invention include embryonic stem cells and adult stem cells. Human embryonic stem cells can be obtained either directly from human embryos or from previously established embryonic stem cell lines (see, e.g., Wobus and Boheler, Physiol. Rev. 85:635, 2003). Stem cells can also be obtained, for example, from umbilical cord blood and adult bone marrow (Rogers and Casper, Human Reproduction Update 9:25, 2003). When possible, such as in the case of stem cells obtained from adult bone marrow, the stem cells can be obtained from the subject to whom the hemangioblasts are later administered or a matched donor. Once isolated, these cells can be expanded in cell culture and treated to induce lycat activity, resulting in differentiation into hemangioblasts.

To cause a stem cell to differentiate into a hemangioblast, lycat activity is induced in the stem cell. This can be achieved by, for example, treatment of the stem cells with an agent found to induce or activate lycat expression (e.g., agents identified using methods described herein). In other examples, induction can be achieved by ex vivo or in vivo gene therapy approaches, such as those described below. Once induced, lycat-expressing cells can be administered to patients suffering from vascular and/or hematopoietic-related diseases, as described above. Hemangioblasts can be further induced to differentiate along a cardiogenic, angiogenic, hemangiogenic, or vasculogenic pathways or lineages, as determined to be appropriate by those of skill in the art.

Lycat-expressing cells can be administered to a subject (e.g., a human subject suffering from an ischemia-related disease or vascular damage) by conventional techniques. For example, the cells can be administered by intravenous infusion. In other examples, the cells are administered directly to a target site (e.g., a limb, the myocardium, or the brain) by, for example, injection of cells in a suitable carrier or diluent, such as a buffered salt solution; by surgical delivery to an internal or external target site (e.g., a limb or a ventricle of the brain); or by use of a catheter directed to a site accessible by a particular blood vessel. In the case of local administration to the brain, the cells can be precisely delivered using stereotactic techniques. Other approaches to local administration involve the use of matrix-cell complexes. Such matrices can include biocompatible (e.g., bioabsorbable) scaffolds, lattices, and/or self-assembling structures, and can be liquid, gel, or solid. The matrices can be pretreated with therapeutic cells so that the matrices are populated with cells in close association to the matrix or its spaces. The cells can adhere to the matrix or can be entrapped or contained within the matrix spaces. The matrix-cell compositions can be introduced into a patient by, for example, implantation, injection, surgical attachment, transplantation with other tissue, or injection.

The cells described above are administered to a subject (e.g., a mammal, such as a human) in an effective amount, which is an amount that produces a desirable result in a treated subject (e.g., increasing vascular growth and/or hematopoiesis in a subject). Such therapeutically effective amounts can be determined empirically by those of skill in the art. Although the range can vary considerably, a therapeutically effective amount may be in the range of from 500 to 1×10⁶ cells per kg body weight of the subject.

Because lycat expression is necessary for hemangioblast development, lycat can be used as a marker for hematopoietic and endothelial precursor cells. Detection of lycat as such a marker can be used in methods for isolating cells expressing lycat (e.g., hemangioblasts) from mixed cell populations (e.g., cord or peripheral blood samples). Isolating cells according to such approaches can be achieved through a number of methods known in the art including, e.g., fluorescence activated cell sorting (FACS), column purification, and methods employing magnetic beads conjugated to anti-lycat antibodies.

### Gene Therapy

As is noted above, lycat genes can be introduced into stem cells ex vivo to generate hemangioblasts that can be used in cell-based therapies. Lycat genes can also be introduced into other cells (e.g., fibroblasts) using ex vivo gene therapy approaches, and the resulting cells implanted into subjects in which the implanted cells produce lycat. As in the case of stem cells used in cell-based therapies, discussed above, the cells used in these ex vivo gene therapy approaches can be, when possible, obtained from the subject to whom the genetically modified cells are later administered or a matched donor. Gene therapy approaches can also be used to introduce lycat genes (e.g., lycat genes included in vectors) into cells in vivo. Numerous methods for gene therapy are well known in the art and can be used in the invention. These approaches can employ vectors such as viral vectors (DNA or RNA) and plasmid vectors, and/or chemical means. Examples of different approaches that can be used in the gene therapy methods of the invention are described as follows.

One example of a viral vector-based gene therapy approach that can be used in the invention employs adeno-associated virus (AAV) vectors, which can achieve latent infection of a broad range of cell types, resulting in persistent expression of a therapeutic gene such as a lycat gene in a subject. As examples, the following AAV vectors can be used: AAV1, AAV2, AAV3, AAV4, AAV5, and AAV6 (Lee et al., Biochem. J. 387:1, 2005). The capsid protein of these vectors can, optionally, be genetically modified, if desired, to direct infection towards a particular tissue type (Lieber, Nature Biotechnology 21:1011, 2003).

Other examples of virus vector-based approaches employ adenoviruses, which infect a wide variety of cell types, including non-dividing cells. The invention includes the use of any one of more than 50 serotypes of adenoviruses that are known in the art, including the most commonly used serotypes for gene therapy: type 2 and type 5. To increase the efficacy of gene expression and prevent unintended spread of the virus, adenoviruses can include genetic modifications, such as E1 region deletions, E1 region and E2 and/or E4 region deletions, or deletion of the entire adenovirus genome except for the cis-acting inverted terminal repeats and a packaging signal (Gardlik et al., Med. Sci. Monit, 11:RA110, 2005). Any adenoviral vectors including such modifications can be used in the invention.

The Invention also includes the use of retroviral vectors including, for example, Moloney Murine Leukemia Virus (MoMLV). These vectors can include genetic modifications including, e.g., deletions of the gag, pol, and/or env genes, as is known in the art. Using retrovirus constructs, gene therapy vectors can be targeted to specific tissues or cells. This can be achieved by the fusion of part of the retrovirus env gene to a sequence encoding the ligand for a tissue-specific receptor. A specific type of retrovirus vector that can be used in the invention is lentivirus vectors, which can infect both proliferating and quiescent cells. An exemplary lentivirus vector for use in gene therapy is HIV-1. Previously constructed genetic modifications of lentiviruses, which can be used in vectors of the present invention, include deletions of all protein encoding genes except those encoding gag, pol, and rev (Moreau-Gaudry et al., Blood 98:2664, 2001).

In addition to the viral vectors described above, other viral vectors that can be used in the invention include, for example, vaccinia virus, bovine papilloma virus, and herpes virus, such as Epstein-Barr Virus vectors. (Also see, for example, the vectors of Miller, Human Gene Therapy 15-14, 1990; Friedman, Science 244:1275-1281, 1989; Eglitis et al., BioTechniques 6:608-614, 1988; Tolstoshev et al., Current Opinion in Biotechnology 1:55-61, 1990; Sharp, The Lancet 337:1277-1278, 1991; Cornetta et al., Nucleic Acid Research and Molecular Biology 36:311-322, 1987; Anderson, Science 226:401-409, 1984; Moen, Blood Cells 17:407-416, 1991; Miller et al., Biotechnology 7:980-990, 1989; Le Gal La Salle et al., Science 259:988-990, 1993; and Johnson, Chest 107:77 S-83S, 1995.)

Gene therapy methods employing chemical means for introducing nucleic acid molecules into cells can also be used in the invention. In one example, cationic liposomes are used. Exemplary cationic liposomes for use in the invention include DOTMA, DOPE, DOSPA, DOTAP, DC-Chol, Lipid GL-67™, and EDMPC. These chemicals can be used individually or in combination to transfect cells with a vector, such as a plasmid, that has been constructed to express a gene of interest such as lycat. Other approaches that can be used in the invention involve the use of lipofection (Felgner et al., Proc. Natl. Acad. Sci. U.S.A. 84:7413, 1987; Ono et al., Neuroscience Letters 17:259, 1990; Brigham et al., Am. J. Med. Sci. 298:278, 1989; Staubinger et al., Methods in Enzymology 101:512, 1983) or asialoorosomucoid-polylysine conjugation (Wu et al., J. Biol. Chem. 263:14621, 1988; Wu et al., J. Biol. Chem. 264:16985, 1989).

In other methods, DNA-polymer conjugates can be used to express a protein of interest, such as lycat, in a patient. In such approaches, a vector constructed to express lycat is combined with a polymer to achieve expression of lycat without the use of a viral vector. Exemplary compounds for use in this approach are polyethyleneimine (PEI), polylysine, polylysine linked to nuclear localization signals, polyamidoamine, and polyarginine (Arg₁₆). Another method of gene therapy that can be used in the invention employs a substantially purified DNA vector (naked DNA) for the expression of lycat in a subject. Such a DNA vector can be administered by injection, use of a gene gun, or electroporation.

In the chemical-based, non-viral approaches described above, the therapeutic material can be directed to certain tissue types. For example, the material can include antibodies, such as multivalent antibodies, receptor ligands, or carbohydrates that direct the materials to the desired tissue.

In the case of ex vivo gene therapy, the vectors described above can be administered directly to cells in culture (e.g., stem cells, such as hematopoietic stem cells, or hemangioblasts), which can be obtained from the patient or from a donor. In addition to such vector-based methods, gene transfer into such cells can be achieved non-vector-based methods such as those described above, as well as transfection methods involving the use of calcium phosphate, DEAE dextran, electroporation, or protoplast fusion.

In general, ex vivo gene therapy results in expression of a therapeutic gene, such as lycat, only in a particular, desired tissue. In such applications, as well as applications in which tissue specific expression of lycat is not a concern, the vectors described above can be constructed so as to constitutively express lycat. Numerous constitutive regulatory elements that can be used in such constructs are well known in the art. For example, certain elements present in the native viruses described above can be used to constitutively express a gene of interest. Other examples of constitutive regulatory elements that dan be used in the invention include β-actin, EF1, EGR1, eIF4A1, FerH, FerL, GAPDH, GRP78, GRP94, HSP70, beta-Kin, ROSA, and ubiquitin B promoters. For in vivo applications, the vectors described above can, if desired, be modified to include regulatory elements that confine the expression of lycat to certain tissue types, such as endothelial or hematopoietic cells. Numerous examples of regulatory elements specific to certain tissue types are well known in the art.

In addition to constitutive and cell/tissue-specific promoters, the gene therapy methods of the invention can employ inducible promoters. In one example of such an approach, cells are transfected with multiple viral or plasmid vectors. Typically a first vector expresses a gene of interest, such as lycat, under the control of a regulatory element that is responsive to the expression product of a second vector. The activity of this expression product is controlled by the addition or administration of a pharmacological compound or other exogenous stimulation. Examples of these systems are those including the following inducing agents or conditions: tetracycline, mifepristone, ponasterone A, papamycin, tamoxifen, radiation, and heat shock (Robson et al., J. Biomed. Biotechnol. 2:110, 2003).

In addition to lycat, the gene therapy approaches described herein can be used to increase levels of other proteins that, similar to lycat, have beneficial effects with respect to the promotion of vascular and hematopoietic growth and differentiation. Thus, gene therapy approaches resulting in increased lycat levels can be carried out in conjunction with approaches used to increase levels of genes such as, for example, Flk1, Flt1, VEGF, Scl, GATA1, c-Myb, and Runx1.

### Protein Therapy

The invention also includes therapeutic methods involving administration of protein preparations of lycat and/or lycat fragments or fusions (see above). In these methods, lycat protein is administered alone or is conjugated to another agent that stabilizes and/or directs localization of the protein. For example, the lycat protein can be conjugated to an agent that facilitates translocation of protein across cell membranes. In one example, protein therapy can involve the use of fusion constructs including a protein of interest, such as lycat, and a protein transduction domain (PTD). The three most commonly used PTDs, which can be used in the invention, are from the Drosophila homeotic transcription protein antennapedia (Antp), the herpes simplex virus structural protein VP22, and the human immunodeficiency virus 1 (HIV-1) transcriptional activator Tat protein (Wadia et al., Curr. Opin. Biotechnol. 13:52, 2002). Also included in the invention are the use of modified PTDs that have enhanced translocation properties (see, e.g., Ho et al., Cancer Res., 61:474, 2001).

Under certain circumstances, it may be desirable to treat patients with lycat through both protein and gene-based therapies. For example, gene therapy resulting in integration into host cells can result in prolonged lycat expression, but it may take some time before therapeutic levels of lycat are reached using this approach alone. To achieve therapeutically effective concentrations of lycat shortly after the initiation of such treatment, before the gene therapy approach results in therapeutic levels, it may be desirable to carry out protein therapy. Using such an approach, therapeutic levels of lycat are obtained both in the short and long term. In other circumstances, only short-term therapy may be desired. In such cases, it may be beneficial to carry out lycat protein therapy alone (i.e., in the absence of lycat gene therapy). In addition to these combinations, lycat protein and/or nucleic acid molecule-based therapies can also be combined with any other type of therapy, such as those described elsewhere herein (e.g., small molecule therapy).

As is discussed further below, standard methods can be used to administer lycat proteins according to the invention. Such approaches include, for example, subcutaneous and intramuscular injection. In other examples, systemic (e.g., intravenous or oral) is used. Other modes of administration, including others described herein, can be selected by those of skill in the art.

### Interfering RNA

The invention also includes the use of interfering RNA (RNAi) in methods to disrupt lycat expression. The term “RNAi” is used herein to refer collectively to several gene silencing techniques, including the use of siRNA (short interfering RNAs), shRNA (short hairpin RNA—an RNA bearing a fold-back stem-loop structure), dsRNA (double-stranded RNA; see, for example, Williams, Biochem. Soc. Trans. 25:509, 1997; Gil and Esteban, Apoptosis 5:107, 2000; Clarke and Mathews, RNA 1:7, 1995; Baglioni and Nilsen, Interferon 5:23, 1983), miRNA (micro RNAs), stRNAs (short (or “small”) temporal RNAs), and the like, all of which can be used in the methods of the present invention.

A number of methods for producing and selecting RNAi molecules, such as shRNAs, siRNAs, and dsRNAs, have been developed and can be used in the present invention (see, e.g., Paddison et al., Methods Mol. Biol. 265:85, 2004; Kakare et al., Appi. Biochem. Biotechnol. 119:1, 2004; Paddison et al., Nature 428:427, 2004; Paddison and Hannon, Cancer Cell 2:17, 2002; Paddison et al., Genes Dev. 16:948, 2002; Hannon and Conklin, Methods Mol. Biol. 257:255, 2004; Katoh et al., Nucleic Acids Res. Suppl. 3:249, 2003; Koper-Emde et al., Biol. Chem. 385:791, 2004; Gupta et al., Proc. Natl. Acad. Sci. U.S.A. 101:1927, 2004; and Paddison et al., Proc. Natl. Acad. Sci. U.S.A. 99:1443, 2002). In addition, commercially available kits can be used to make RNAi for use in the methods of the invention (e.g. GeneEraser™ (catalog #240090) from Stratagene, La Jolla, Calif.). For further descriptions of this technology, see, for example, Brumelkamp et al., Sciencexpress, Mar. 21, 2002; Sharp, Genes Dev. 13:139, 1999; Cathew, Curr. Op. Cell Biol. 13:244, 2001; Zamore et al., Cell 101:25, 2000; Bass, Nature 411:428, 2001; Elbashir et al., Nature 411:494, 2001; PCT Publication Nos. WO 00/44895, WO 01/36646, WO 99/32619, WO 00/01846, WO 01/29058, WO 99/07409, and WO 00/44914, Allshire, Science 297:1818, 2002; Volpe et al., Science 297:1833, 2002; Jenuwein, Science 297:2215, 2002; Hall et al., Science 297:2232, 2002; Hutvagner and Zamore, Science 297:2056, 2002; McManus et al., RNA 8:842, 2002; Reinhart et al., Genes Dev. 16:1616, 2002; and Reinhart and Bartel, Science 297:1831, 2002.

Lycat nucleic acid molecules as described herein can be used as guide sequences in the design of RNAi molecules of the invention, which can include sense and/or antisense sequences or regions that are generally covalently linked by nucleotide or non-nucleotide linker molecules, as is known in the art. Alternatively, the linkages can be non-covalent, involving, for example, ionic, hydrogen bonding, Van der Waals, hydrophobic, and/or stacking interactions. siRNAs of the invention can be, e.g., between 19 and 29 nucleotides in length, while dsRNAs can be at least 30, 50, 100, or 500 nucleotides in length. As is known in the art, shRNAs are generally designed to form double-stranded regions of 19 to 29 nucleotides in length, although these lengths can vary (see Paddison et al., Genes Dev. 16:948, 2002). Exemplary requirements for siRNA length, structure, chemical composition, cleavage site position, and sequences essential to mediate efficient RNAi activity are described, for example, by Elbashir et al., EMBO J. 20:6877, 2001; and Nykanen et al., Cell 107:309, 2001.

RNAi molecules of the present invention include any form of RNA, such as partially purified RNA, essentially pure RNA, synthetic RNA, recombinantly produced RNA, as well as RNA that differs from naturally occurring RNA by the addition, deletion, substitution, and/or alteration of one or more nucleotides. Such alterations can include the addition of non-nucleotide material to, e.g., the end(s) of the RNA or internally (at one or more nucleotides of the RNA), or the RNA molecule can contain a 3′hydroxyl group. Nucleotides in the RNAi molecules of the present invention can also include non-standard nucleotides, including non-naturally occurring nucleotides or deoxyribonucleotides. Examples of modified nucleotides that can be included in RNAi molecules of the invention, such as 2′-O-methyl ribonucleotides, 2′-deoxy-2′-fluoro ribonucleotides, “universal base” nucleotides, 5-C-methyl nucleotides, nucleotides with phosphorothioate internucleotide linkages, and inverted deoxyabasic residues, are described, for example, in U.S. Patent Application Publication No. 20040019001. RNAi molecules directed against lycat can be used individually, or in combination with other RNAi constructs, for example, constructs against Flk1, Flt1, VEGF, Scl, GATA1, c-Myb, and Runx1.

### Screening Methods

The invention also includes methods for identifying compounds (e.g., small organic or inorganic molecules) that can be used to treat the diseases or conditions described herein, whether by direct administration or by use in differentiating stem cells into hemangioblasts ex vivo, as described above. Compounds that are found to increase lycat levels, stability, activity, and/or expression can be used to treat conditions that would benefit from increased vascularization or hematopoiesis (see above), while compounds that decrease lycat levels, stability, activity, and/or expression can be used to treat conditions in which it is beneficial to decrease blood vessel growth and/or hematopoiesis.

In one example of a screening method of the invention, a zebrafish having a mutation in a lycat gene (e.g., a zebrafish having the cloche mutation described herein; see below) is contacted with a candidate compound, and the effect of the compound on the phenotype of the zebrafish is monitored relative to an untreated, identically mutant control. The zebrafish can be treated early in development, before the cloche phenotype is apparent, at a stage during development when the phenotype is apparent, or at maturity. After a compound has been shown to have a desired effect in the zebrafish system, it can be tested in other animal models, for example, in mice or other animals having a lycat gene mutation. Alternatively, testing in such animal model systems can be carried out in the absence of zebrafish testing.

In addition to animal models, screening assays can be carried out using in vitro or cell culture-based systems. Any number of methods can be used for carrying out such screening assays, including high throughput techniques. According to one approach, candidate compounds are added at varying concentrations to the culture medium of cells (e.g., stem cells) that may or may not be expressing lycat. Gene expression is then measured by, for example, use of a cDNA microarray, quantitative reverse-transcription polymerase chain reaction (qRT-PCR), or standard Northern blot analysis. The effects of candidate compounds can also be measured at the level of polypeptide production in a cell using methods including standard immunological techniques (e.g., ELISA, RIA, flow cytometry, western blotting, and immunoprecipitation) employing an antibody specific for lycat. The level of gene expression in the presence of the candidate compound is compared to the level measured in a control culture medium lacking the candidate molecule. In other examples, reporter constructs can be used to indirectly measure lycat expression. In such methods, the expression of a reporter molecule (e.g., luciferase, green fluorescent protein, or β-galactosidase) is placed under the control of the regulatory region upstream of lycat as described herein. A compound that increases lycat expression, stability, or expression can be used to stimulate vascular growth and/or hematopoiesis, as described herein, while a compound that decreases lycat expression, stability, or expression can be used, for example, to treat a proliferative disorder (e.g., cancer).

Further, compounds can be screened for inhibition of the acyltransferase activity of lycat. Acyltransferase activity can be screened by enzymatic assays well known in the art including fluorometric assays, radioactive assays, and immunological assays. In one example of such an assay, the ability of lycat to transfer labeled palmitate to a substrate can be measured. An exemplary substrate for an acyltransferase assay is VEGF. Exemplary methods for detecting palmitoylation of VEGF are described further. These methods can also be used to measure the acyltransferase activity of lycat with additional substrates.

Candidate compounds can also be screened for those that specifically bind to and modulate lycat activity. The efficacy of such a candidate compound can be dependent upon its ability to interact with lycat. Such an interaction can be detected using any of a number of standard binding techniques and functional assays. For example, compounds that bind to lycat can be identified using chromatography-based techniques. In one example, recombinant lycat is purified by standard techniques from cells engineered to express lycat and immobilized on a column. A solution of candidate compounds is then passed through the column, and a compound specific for lycat is identified on the basis of its ability to bind to lycat and be immobilized on the column. To isolate the compound, the column is washed to remove non-specifically bound molecules, and the compound of interest is then eluted from the column and collected. Compounds that are identified as binding to lycat with an affinity constant less than or equal to 10 mM may be considered particularly useful in the invention.

Once found to have a specific effect on lycat expression, stability, and/or activity, or to impact a phenotype associated with altered lycat expression, stability, and/or activity, a compound can be tested in model systems for efficacy with respect to phenotypes of diseases or conditions described herein. For example, the compounds can be tested for their impact on vascularization and/or hematopoietic in animal model systems, or can be tested for their impact on cellular proliferation in cell culture or animal based systems.

Potential lycat agonists or antagonists can be identified from, e.g., libraries of natural products, synthetic (or semi-synthetic) extracts, and chemical libraries using methods that are well known in the art. Candidate compounds to be tested include purified (or substantially purified) molecules or one or more components of a mixture of compounds (e.g., an extract or supernatant obtained from cells; Ausubel et al., Current Protocols in Molecular Biology, Vol. 2, 1994), and such compounds further include both naturally occurring or artificially derived chemicals and modifications of existing compounds. For example, candidate compounds can be polypeptides, synthesized organic or inorganic molecules, naturally occurring organic or inorganic molecules, nucleic acid molecules, and components thereof.

Numerous sources of naturally occurring candidate compounds are readily available to those skilled in the art. For example, naturally occurring compounds can be found in cell (including plant, fungal, prokaryotic, and animal) extracts, mammalian serum, growth medium in which mammalian cells have been cultured, protein expression libraries, or fermentation broths. In addition, libraries of natural compounds in the form of bacterial, fungal, plant, and animal extracts are commercially available from a number of sources, including Biotics (Sussex, UK), Xenova (Slough, UK), Harbor Branch Oceanographic Institute (Ft. Pierce, Fla.), and PharmaMar, U.S.A. (Cambridge, Mass.). Further, libraries of natural compounds can be produced, if desired, according to methods that are known in the art, e.g., by standard extraction and fractionation.

Artificially derived candidate compounds are also readily available to those skilled in the art. Numerous methods are available for generating random or directed synthesis (e.g., semi-synthesis or total synthesis) of any number of chemical compounds, including, for example, saccharide-, lipid-, peptide-, and nucleic acid molecule-based compounds. In addition, synthetic compound libraries are commercially available from Brandon Associates (Merrimack, N.H.) and Aldrich Chemicals (Milwaukee, Wis.). Libraries of synthetic compounds can also be produced, if desired, according to methods known in the art, e.g., by standard extraction and fractionation. Furthermore, if desired, any library or compound can be readily modified using standard chemical, physical, or biochemical methods. The techniques of modern synthetic chemistry, including combinatorial chemistry, can also be used (reviewed in Schreiber, Bioorganic and Medicinal Chemistry 6:1172-1152, 1998; Schreiber, Science 287:1964-1969, 2000).

When a crude extract is found to have a desired effect, further fractionation of the positive lead extract can be carried out to isolate chemical constituents responsible for the observed effect. Thus, the goal of the extraction, fractionation, and purification process is the careful characterization and identification of a chemical entity within the crude extract having a desired activity. The same assays described herein for the detection of activities in mixtures of compounds can be used to purify the active component and to test derivatives of these compounds. Methods of fractionation and purification of such heterogeneous extracts are well known in the art. If desired, compounds shown to be useful agents for treatment can be chemically modified according to methods known in the art.

### Antibody Production

The invention also provides methods of generating antibodies (e.g., monoclonal, polyclonal, poly-specific, or mono-specific antibodies) against lycat, which can be used for diagnostic, research, or therapeutic purposes. Numerous methods for making antibodies are known in the art and can be used in the invention to make such antibodies. In one example, a coding sequence for a lycat peptide or polypeptide is expressed as a C-terminal fusion with glutathione S-transferase (GST) (Smith et al., Gene 67:31, 1988). The fusion protein is purified on glutathione-Sepharose beads, eluted with glutathione, cleaved with thrombin (at an engineered cleavage site), and purified for immunization of rabbits. Primary immunizations are carried out with Freund's complete adjuvant and subsequent immunizations with Freund's incomplete adjuvant. Antibody titers are monitored by Western blot and immunoprecipitation analyses using the thrombin-cleaved protein fragment of the GST fusion protein. Immune sera are affinity purified using CNBr-Sepharose-coupled protein. Antiserum specificity can be determined using a panel of unrelated GST proteins.

As an alternate or adjunct immunogen to GST fusion proteins, peptides corresponding to relatively unique immunogenic regions of a polypeptide of the invention can be generated and coupled to keyhole limpet hemocyanin (KLH) through an introduced C-terminal lysine. Antiserum to each of these peptides is similarly affinity purified on peptides conjugated to BSA, and specificity is tested by ELISA or Western blot analysis using peptide conjugates, or by Western blot or immunoprecipitation using the polypeptide expressed as a GST fusion protein.

Alternatively, monoclonal antibodies that specifically bind lycat can be prepared using standard hybridoma technology (see, e.g., Kohler et al., Nature 256:495, 1975; Kohler et al., Eur. J. Immunol. 6:511, 1976; Kohler et al., Eur. J. Immunol. 6:292, 1976; Hammerling et al., Monoclonal Antibodies and T Cell Hybridomas, Elsevier, N.Y., 1981). Once produced, monoclonal antibodies can also be tested for specific recognition by Western blot or immunoprecipitation analysis. Antibodies that specifically recognize lycat can be used, for example, in immunoassays. Alternatively monoclonal antibodies can be prepared using the polypeptide of the invention described above and a phage display library (Vaughan et al., Nature Biotech. 14:309, 1996).

Antibodies of the invention can, optionally, be produced using fragments of the lycat polypeptide that lie outside generally conserved regions (e.g., the acyltransferase domain) and appear likely to be antigenic by criteria such as high frequency of charged residues. In one specific example, such fragments are generated by standard techniques of PCR and cloned into the pGEX expression vector. Fusion proteins are expressed in E. coli and purified using a glutathione agarose affinity matrix. To minimize potential problems of low affinity or specificity of antisera, two or three such fusions are generated for each protein, and each fusion is injected into at least two rabbits. Antisera are raised by injections in a series, and can include, for example, at least three booster injections.

In addition to intact monoclonal and polyclonal anti-lycat antibodies, the invention also includes various genetically engineered antibodies, humanized antibodies, chimeric antibodies, and antibody fragments, including F(ab′)2, Fab′, Fab, Fv, and sFv fragments. Truncated versions of monoclonal antibodies, for example, can be produced by recombinant methods in which plasmids are generated that express the desired monoclonal antibody fragment(s) in a suitable host. Antibodies can be humanized by methods known in the art, e.g., monoclonal antibodies with a desired binding specificity can be commercially humanized (Scotgene, Scotland; Oxford Molecular, Palo Alto, Calif.). Fully human antibodies, such as those expressed in transgenic animals, are also included in the invention (Green et al., Nature Genetics 7:13-21, 1994).

Examples of approaches that can be used to generate antibodies of the invention include the following. Ladner (U.S. Pat. Nos. 4,946,778 and 4,704,692) describes methods for preparing single polypeptide chain antibodies. Ward et al., Nature 341:544-546, 1989, describes the preparation of heavy chain variable domains, which they term “single domain antibodies,” and which have high antigen-binding affinities. McCafferty et al., Nature 348:552-554, 1990, shows that complete antibody V domains can be displayed on the surface of fd bacteriophage, that the phage bind specifically to antigen, and that rare phage (one in a million) can be isolated after affinity chromatography. Boss et al., U.S. Pat. No. 4,816,397, describes various methods for producing immunoglobulins, and immunologically functional fragments thereof, that include at least the variable domains of the heavy and light chains in a single host cell. Cabilly et al., U.S. Pat. No. 4,816,567, describes methods for preparing chimeric antibodies.

Antibodies to lycat can be used, as noted above, to detect lycat or to inhibit the biological activities of lycat. For example, a nucleic acid molecule encoding an antibody or portion of an antibody can be expressed within a cell to inhibit lycat function. In addition, the antibodies can be coupled to compounds, such as radionuclides and liposomes, for diagnostic or therapeutic uses. Antibodies that inhibit the activity of a lycat polypeptide described herein can also be useful in preventing or slowing the development of a disease caused by inappropriate expression of a wild type or mutant lycat gene.

### Administration of Therapeutic Agents

Any appropriate route of administration can be employed to administer a compound identified as described above, a lycat gene, protein, RNAi, or antibody, according to the invention. For example, administration can be parenteral, intravenous, intra-arterial, subcutaneous, intramuscular, intraventricular, intracapsular, intraspinal, intracisternal, intraperitoneal, intranasal, by aerosol, by suppository, or oral.

A therapeutic compound of the invention can be administered within a pharmaceutically acceptable diluent, carrier, or excipient, in unit dosage form. Administration can begin before or after the patient is symptomatic. Methods that are well known in the art for making formulations are found, for example, in Remington's Pharmaceutical Sciences (18^{th} edition), ed. A. Gennaro, 1990, Mack Publishing Company, Easton, Pa.

Therapeutic formulations can be in the form of liquid solutions or suspensions. Formulations for parenteral administration can contain, for example, excipients, sterile water, or saline; polyalkylene glycols, such as polyethylene glycol; oils of vegetable origin; or hydrogenated napthalenes. Biocompatible, biodegradable lactide polymer, lactide/glycolide copolymer, or polyoxyethylene-polyoxypropylene copolymers can be used to control the release of the compounds. Other parenteral delivery systems that can be used include ethylene-vinyl acetate copolymer particles, osmotic pumps, implantable infusion systems, and liposomes. For oral administration, formulations can be in the form of tablets or capsules. Formulations for inhalation can contain excipients, for example, lactose, or can be aqueous solutions containing, for example, polyoxyethylene-9-lauryl ether, glycocholate, and deoxycholate, or can be oily solutions for administration in the form of nasal drops or as a gel. Alternatively, intranasal formulations can be in the form of powders or aerosols.

### Diagnostic Methods

Nucleic acid molecules encoding lycat proteins, as well as polypeptides encoded by these nucleic acid molecules and antibodies specific for these polypeptides, can be used in methods to diagnose or to monitor diseases and conditions involving mutations in, or inappropriate expression of, genes encoding lycat.

The diagnostic methods of the invention can be used, for example, with patients that have a disease or condition that may be associated with lycat, in an effort to determine its etiology and, thus, to facilitate selection of an appropriate course of treatment. The diagnostic methods can also be used with patients who have not yet developed, but who are at risk of developing, such a disease or condition, or with patients that are at an early stage of developing such a disease or condition. Also, the diagnostic methods of the invention can be used in prenatal genetic screening, for example, to identify parents who may be carriers of a recessive mutation in a gene encoding a lycat protein. In addition, the methods can be used to investigate whether a lycat mutation may be contributing to a disease or condition (e.g., a disease of the vasculature, e.g., heart disease, or a disease or condition relating to hematopoiesis) in a patient, by determining whether a lycat gene of a patient includes a mutation. The methods of the invention can be used to diagnose (or to prevent or treat) the disorders described herein in any mammal, for example, in humans, domestic pets, or livestock.

Abnormalities in lycat that can be detected using the diagnostic methods of the invention include those characterized by, for example, (i) a gene encoding a lycat protein containing a mutation that results in the production of an abnormal lycat protein, (ii) an abnormal lycat polypeptide itself (e.g., a truncated protein), and (iii) a mutation in a lycat gene that results in production of an abnormal amount of this protein. Detection of such abnormalities can be used to diagnose human diseases or conditions related to lycat, such as those affecting the vasculature. Exemplary of the mutations in lycat genes is the cloche mutation, which is described further below.

A mutation in a lycat gene can be detected in any tissue of a subject, even one in which this protein is not expressed. Because of the possibly limited number of tissues in which these proteins may be expressed, for limited time periods, and because of the possible undesirability of sampling such tissues (e.g., heart tissue) for assays, it may be preferable to detect mutant genes in other, more easily obtained sample types, such as in blood or amniotic fluid samples.

Detection of a mutation in a gene encoding a lycat protein can be carried out using any standard diagnostic technique. For example, a biological sample obtained from a patient can be analyzed for one or more mutations (e.g., a cloche mutation) in nucleic acid molecules encoding a lycat protein using a mismatch detection approach. Generally, this approach involves polymerase chain reaction (PCR) amplification of nucleic acid molecules from a patient sample, followed by identification of a mutation (i.e., a mismatch) by detection of altered hybridization, aberrant electrophoretic gel migration, binding, or cleavage mediated by mismatch binding proteins, or by direct nucleic acid molecule sequencing. Any of these techniques can be used to facilitate detection of a mutant gene encoding a lycat protein, and each is well known in the art. For instance, examples of these techniques are described by Orita et al. (Proc. Natl. Acad. Sci. U.S.A. 86:2766-2770, 1989) and Sheffield et al. (Proc. Natl. Acad. Sci. U.S.A. 86:232-236, 1989).

As noted above, in addition to facilitating diagnosis of an existing disease or condition, mutation detection assays also provide an opportunity to diagnose a predisposition to disease related to a mutation in a lycat gene before the onset of symptoms. For example, a patient who is heterozygous for a gene encoding an abnormal lycat protein (or an abnormal amount thereof) that suppresses normal lycat biological activity or expression may show no clinical symptoms of a disease related to such proteins, and yet possess a higher than normal probability of developing such disease. Given such a diagnosis, a patient can take precautions to minimize exposure to adverse environmental factors, and can carefully monitor their medical condition, for example, through frequent physical examinations. As mentioned above, this type of diagnostic approach can also be used to detect a mutation in a gene encoding the lycat protein in prenatal screens.

While it may be preferable to carry out diagnostic methods for detecting a mutation in a lycat gene using genomic DNA from readily accessible tissues, as noted above, mRNA encoding this protein, or the protein itself, can also be assayed from tissue samples in which it is expressed. Expression levels of a gene encoding lycat in such a tissue sample from a patient can be determined by using any of a number of standard techniques that are well known in the art, including northern blot analysis and quantitative PCR (see, e.g., Ausubel et al., supra; PCR Technology: Principles and Applications for DNA Amplification, H. A. Ehrlich, Ed., Stockton Press, NY; Yap et al. Nucl. Acids. Res. 19:4294, 1991).

In another diagnostic approach of the invention, an immunoassay is used to detect or to monitor the level of a lycat protein in a biological sample. Polyclonal or monoclonal antibodies specific for the lycat protein can be used in any standard immunoassay format (e.g., ELISA, Western blot, or RIA; see, e.g., Ausubel et al., supra) to measure polypeptide the levels of lycat. These levels can be compared to levels of lycat in a sample from an unaffected individual. Detection of a decrease in production of lycat using this method, for example, may be indicative of a condition or a predisposition to a condition involving insufficient biological activity of the lycat protein.

Immunohistochemical techniques can also be utilized for detection of lycat protein in patient samples. For example, a tissue sample can be obtained from a patient, sectioned, and stained for the presence of lycat using an anti-lycat antibody and any standard detection system (e.g., one that includes a secondary antibody conjugated to an enzyme, such as horseradish peroxidase). General guidance regarding such techniques can be found in, e.g., Bancroft et al., Theory and Practice of Histological Techniques, Churchiil Livingstone, 1982, and Ausubel et al., supra.

### Experimental Results

The first blood and vessel cells in the embryo are believed to derive from a common progenitor, termed the “hemangioblast.” Experiments are described below which show that lysocardiolipin acyltransferase, lycat (also referred to herein as “cloche” and “clo”), is critical for the establishment of both hematopoietic and endothelial lineages. In summary, lycat is expressed prior to blood and vessel cell formation in the intermediate cell mass of the zebrafish embryo, and subsequently in endothelium and mature blood cells. Reduction of lycat levels abolishes both endothelial and hematopoietic lineages. Lycat rescues endothelial and blood lineages otherwise absent in zebrafish cloche mutant embryos. In murine embryoid bodies, lycat expression causes expansion of hematopoietic (CD45⁺) and endothelial (Flk1⁺) populations. Hence, the acyltransferase lycat appears to define the hemangioblast and to be essential for both blood and endothelial lineages.

The hematopoietic stem cell gives rise to the entire panoply of bone-marrow-derived cells, including erythrocytes, monocytes, macrophages, T cells, B cells, and natural killer cells (Reya et al., Nature 414:105, 2001; Orkin, Nat. Rev. Genet. 1:57, 2000). For many of these lineages, the genetic pathway has been elucidated (Orkin, Nat. Rev. Genet. 1:57, 2000). Evidence for a common precursor cell, termed the “hemangioblast,” includes the spatial contiguity of blood cells and blood vessels in the early embryo (Coultas et al., Nature 438:937, 2005), the ability of the blast colony forming cell to generate both cell types (Huber et al., Nature 432:625, 2004; Choi et al., Development 125:725, 1998; Kennedy et al., Nature 386:488, 1997), overlapping expression of numerous genes in both lineages, the impaired formation of both lineages from particular mutations in the mouse (Flk1, VEGF) (Shalaby et al., Cell 89:981, 1997; Damert et al., Development 129:1881, 2002; Martin et al., Development 131:693, 2004; Ferrera et al., Nature 380:439, 1996), and zebrafish (cloche) (Stainier et al., Development 121:3141, 1995; Stainier et al., Development 123:285, 1996), and the ability of the Scl gene to induce expression of genes specific for both endothelium and blood cells (Gering et al., EMBO J. 17:4029, 1998).

The first blood cells of the vertebrate embryo derive from the mesoderm (extraembryonic mesoderm in mice and posterior lateral mesoderm (PLM) in zebrafish) (Huber et al., Nature 432:625, 2004; Orkin et al., Annu. Rev. Genet. 31:33, 1997). At this early stage, the blood cells primarily consist of red blood cells. Subsequently, long term hematopoietic stem cells arise in the aortal-gonadal-mesonephros (AGM) region, possessing the same multilineage capacity as do stem cells derived from the bone marrow (Orkin, Nat. Rev. Genet. 1:57, 2000; Orkin et al., Annu. Rev. Genet. 31:33, 1997). For embryonic circulation to be established, the endothelial cells that constitute the early vessels must be generated simultaneously with blood. Several lines of investigation suggest a shared common precursor between the earliest embryonic endothelium and blood cells (Coultas et al., Nature 438:937, 2005; Choi et al., Development 125:725, 1998; Kennedy et al., Nature 386:488, 1997; Shalaby et al., Cell 89:981, 1997).

### A. Identification of Lycat

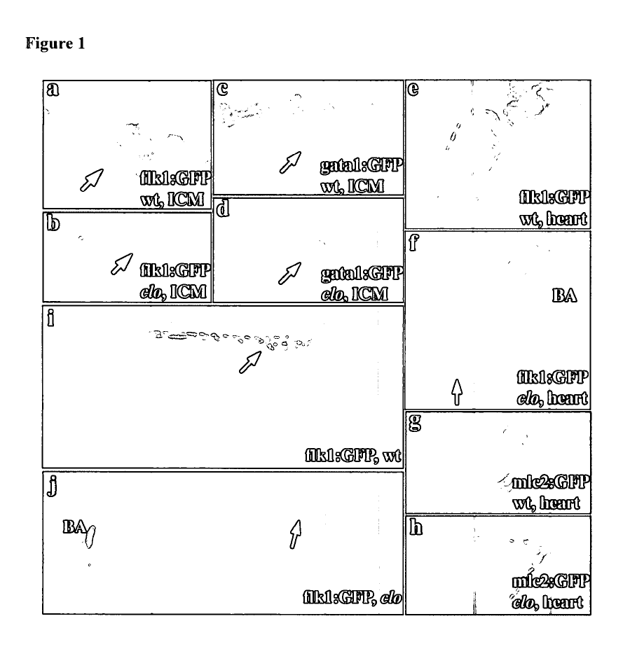

In large-scale genetic screens for cardiovascular mutants in zebrafish, the do mutant was isolated with three alleles called clo^{m39}, clo^{mv087b} and clo^{m378}) (Stainier et al., Development 123:285, 1996; Stainier et al., Development 121:3141, 1995). The do mutant lacks endocardium, vascular endothelial cells, and hematopoietic cells, probably by affecting the formation of hemangioblasts (Stainier et al., Development 121:3141, 1995). All three alleles are fully penetrant in different genetic backgrounds, and transheterozygous embryos among them display the same phenotypes as homozygotes for each single mutant allele. Epistasis experiments suggest that do acts upstream of Flk1 and Scl (Fouquet et al., Dev. Biol. 183:37, 1997; Liao et al., Genes Dev. 12:621, 1998; Liao et al., Development 124:381, 1997), but not VEGF-A (Liang et al., Mech. Dev. 108:29, 2001), at the transcriptional level. Scl can specify the hemangioblast formation from early lateral mesoderm at the expense of other mesodermal fates as seen when applying gain-of-function analysis in zebrafish (Gering et al., EMBO J. 17:4029, 1998; Gering et al., Development 130:6187, 2003). We further characterized the clo mutant phenotypes using transgenic GFP markers under control of zebrafish tissue- or cell-specific promoter and enhancer elements, including the Flk1 promoter to direct GFP expression in vascular endothelial cells and the endocardium, the GATA1 promoter to direct GFP expression in hematopoietic stem cells, and the MLC2 promoter to direct GFP expression in cardiac myocytes. We have established several zebrafish clo^{mv087b} lines with a transgene Flk1:GFP called TG[Flk1:GFP], GATA1:GFP called TG[GATA1:GFP], and MLC2:GFP called TG[MLC2:GFP] (Cross et al., ArterioScler. Thromb. Vasc. Biol. 23:911, 2003; Long et al., Proc. Natl. Acad. Sci. U.S.A. 94:6267, 1997; Rottbauer et al., Cell 27:661, 2002). It was shown that GFP expression patterns in wild-type TG[Flk1:GFP] embryos, recapitulate that of Flk1 transcripts using RNA in situ analysis (Cross et al., ArterioScler. Thromb. Vasc. Biol. 23:911, 2003). At 15 hpf, we observed two stripes of GFP-positive cells located in the lateral mesoderm of wild-type TG[Flk1:GFP] embryos but not in homozygous clo TG[Flk1:GFP] embryos. At 24 hpf, GFP marks the blood islands, dorsal aorta, axial vein, and inter-segmental vessels of wild-type TG[Flk1:GFP] embryos (FIG. 1a and 1i), but very few GFP-positive cells are located in the blood islands of homozygous do mutant embryos (FIGS. 1b and 1j). We also found significantly diminished GFP-positive cells in the blood islands of homozygous do mutant embryos with TG[GATA1:GFP] (FIG. 1d), as compared to that of wild-type TG[GATA1:GFP] embryos (FIG. 1c). These observations have substantiated the previous report that do is essential for both vascular and hematopoietic cell lineages in zebrafish (Stainier et al., Development 121:3141, 1995). Using transgenic GFP marking techniques, we further observed the endocardium-specific GFP expression in wild-type TG[Flk1:GFP] embryos (FIG. 1e), but not in homozygous do mutant embryos (FIG. 1f; the arrow points to the heart). There are comparable GFP signals in the myocardium of both homozygous clo TG[MLC2:GFP] embryos and wild-type TG[MLC2:GFP] embryos (FIGS. 1g and 1h). Therefore, clo is absolutely required for the formation of the endocardium, but not the myocardium.

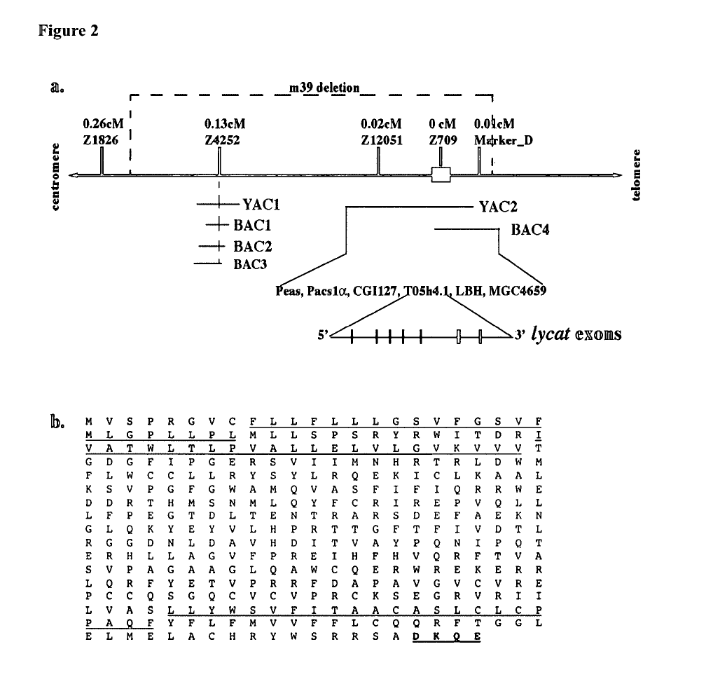

Although cloche was first described over a decade ago, its molecular nature has remained elusive. We mapped cloche to chromosome 13 (FIG. 2a). The clo^{m39} allele deletes a region of approximately 0.2 cM telomeric to marker Z1826. The clo^{m39} allele exhibits the same phenotype of ENU-induced cloche alleles, including clo^{m378} and clo^{fv087b}. A high-resolution genetic map of the clo^{fv087b} allele identified six candidate genes within this deletion (FIG. 2a). Antisense morpholinos targeting each candidate identified only one gene that could recapitulate the cloche phenotype. This gene encodes the zebrafish lycat with a putative endoplasmic reticulum retention signal at its C-terminus (FIG. 2b).

Since lycat occurs within the 0.20 cM interval deleted in clo^{m39} mutant embryos, we were interested in whether lycat is cloche and if it lies within the critical interval of clo^{fv087b} using high-resolution mapping (FIG. 2a). Sequencing of the cloche alleles (clo^{fv087b} and clo^{m378}) does not reveal any clear causal mutation, and maternal transcript in the early embryo obscures any possible diminution that might occur were its expression to be abnormally regulated. In addition, zebrafish lycat and the surrounding loci are imbedded in highly repetitive sequences that make finding valid point mutations very challenging. Since the existence of critical enhancer or repressor elements can exist well outside of the coding region of a gene, the location of mutations could be many kilobases away. An example of long-distance regulators in hematopoietic gene expression is the HS elements that are 10-50 kb away from the developmentally regulated globin gene cluster (Tuan et al., Proc. Natl. Acad. Sci. U.S.A. 86(8):2554-2558, 1989). Distant point mutations in regulatory elements can alter developmental fate. For example, in C. elegans, a point mutation within a 9 bp TRA-1A repressor binding site that prevents the binding of TRA-1A was located 5.6 kb downstream of the egl-1 coding sequence (Conradt and Horvitz, Cell 98(3):317-327, 1999). The identification of conserved regions between zebrafish and Fugu, mouse and humans could pinpoint critical cloche regulatory elements. One could then verify important regions by transgenic rescue of cloche mutants with various portions of the upstream, downstream, or even intronic sequences ligated to the lycat cDNA.

Our studies show that the novel acyltransferase lycat is the long sought cloche gene. Acyltransferases, such as porcupine and skinny hedgehog, are key signaling regulators during development (Nusse, Development 130:5297, 2003). The cardiolipin modifier, lysocardiolipin acyltransferase (Lycat/Alcat1), is strongly expressed in the mouse heart, but its developmental role is unknown (Cao et al., J. Biol. Chem. 279:31727, 2004). We examined this gene because, as discussed above, it localized to the interval deleted in cloche (clo^{m39}) mutant embryos (FIG. 2a). The zebrafish protein is 62% identical and 78% similar to the mouse protein with conservation of the acyltransferase motif and the ER retention signal on the C-terminus (FIG. 2b).

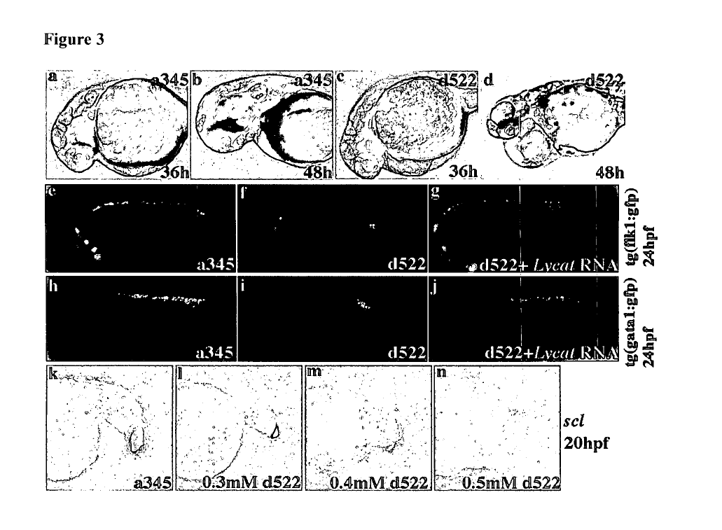

The functional evidence that lycat controls both hematopoietic and endothelial lineages is shown by experimental reduction of lycat levels in zebrafish embryos. Embryos injected with three independent antisense morpholinos lack blood cells, blood vessels, and endocardium (FIGS. 3a to 3d; Table 1). Flk1⁺ and GATA1⁺ cells are dramatically reduced when zebrafish lycat expression is reduced by antisense morpholino targeting in Tg(flk1:GFP) or Tg(gata1:GFP) transgenic embryos (FIGS. 3f and 3i). 88% (132/150) of Tg(flk1:GFP) embryos microinjected with d522 (lycat morpholino targeting the exon 2 splice donor site), lack blood cells, vascular endothelium, and endocardium (Table 1b). The lycat morpholino also reduces expression of Scl in the intermediate cell mass (ICM) in a dose-dependent manner (FIGS. 3k to 3n). Mouse lycat mRNA rescues the morpholino-associated reduction of Flk1⁺ and GATA1⁺ cells (FIGS. 3g and 3j; Table 1), indicating that the lycat genes are functional orthologues.

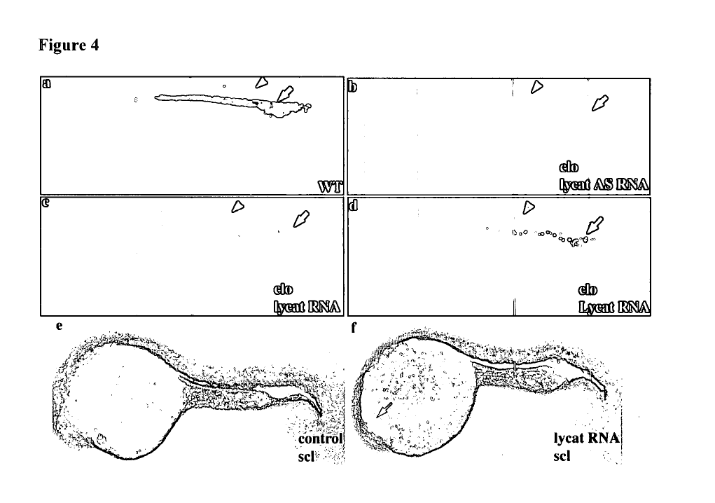

We examined whether lycat can rescue cloche mutant embryos, which lack blood cells and endothelium, and do not express the early hematopoietic genes, such as Scl and gata1. We used clo^{fv087b/+};Tg(gata1:GFP) embryos to track the earliest blood forming areas. Injection of these embryos with lycat RNA dramatically increases GFP expression in the blood forming areas of cloche mutants (FIGS. 4c and 4d). lycat mRNA injection rescues 38.5% (35/91) of cloche mutant embryos. Phenotypically wild-type but genotypically cloche mutants are confirmed by PCR genotyping using Marker D (FIG. 4a, Table 1).

Scl is believed to be involved in hemangioblast function because its overexpression expands hematopoietic and endothelial populations in zebrafish embryos (Gering et al., EMBO J. 17:4029, 1998), and Scl deficient ES cells can not generate blast colony-forming cells in embryoid bodies (Faloon et al., Development 127:1931, 2000; Robertson et al., Development 127:2447, 2000). Therefore, we examined the effect of lycat on Scl expression. Microinjection of zebrafish lycat RNA into embryos increases Scl-expressing cells in the ICM (FIGS. 4e and 4f), and induces ectopic Scl-expression in the head and anterior part of embryo (FIG. 4f, arrow). Ectopic gata1:GFP in the anterior part of embryo was also observed in the rescued cloche embryo (FIG. 4c). Therefore, zebrafish lycat can instructively increase the formation of the Scl⁺ hemangioblast lineage in zebrafish embryos.

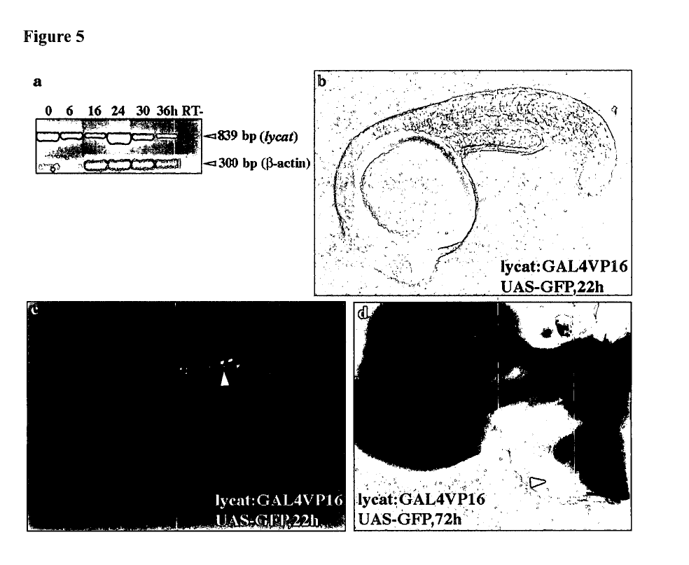

Zebrafish lycat mRNA is detected in fertilized eggs and is throughout the early embryo (FIG. 5a). This presumptive maternal transcript interferes with the ability to detect tissue-specific zygotic expression. To examine the zygotic pattern of zebrafish lycat expression, we isolated a genomic DNA fragment immediately upstream of the predicted ATG start codon, and used it to drive the expression of a Gal4-VP16 fusion protein (Koster et al., Dev. Biol. 233:329, 2001). Co-injection of zebrafish lycat:GAL4-VP16 and a UAS-GFP plasmid results in selective GFP expression in the posterior lateral mesoderm (PLM) during gastrulation and in the ICM and nascent vasculature during somitogenesis (FIG. 5c). Thereafter, GFP expression persists in circulating blood cells and the endocardium of the heart (FIG. 5d).

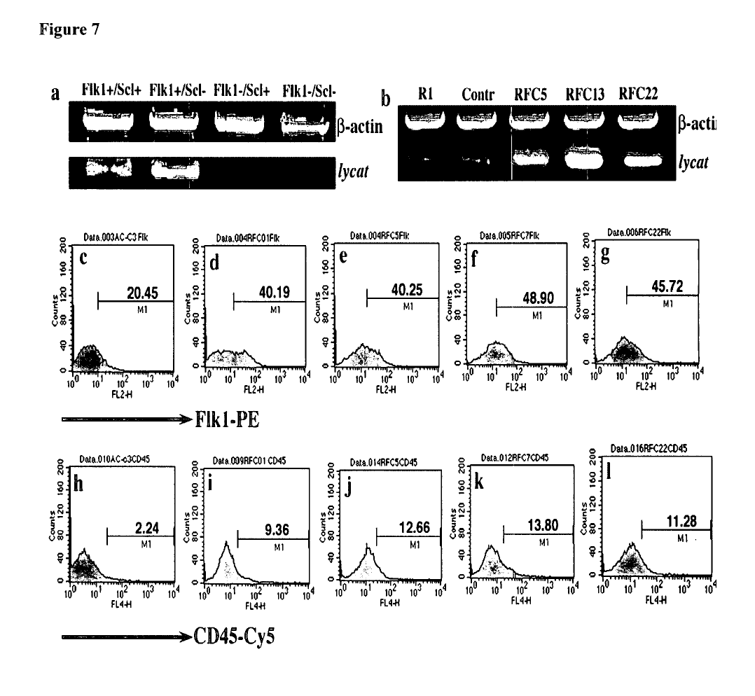

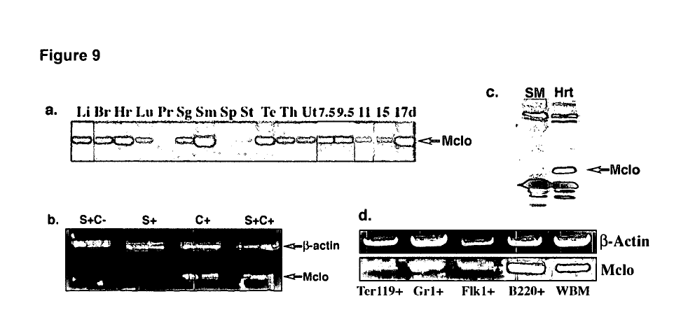

We examined the role of lycat in lineage commitment using the mouse embryonic stem (ES) cell in vitro differentiation system that has been successful for the study of hematopoietic and endothelial lineage development (Choi et al., Development 125:725, 1998; Wiles et al., Development 111:259, 1991). Lycat is expressed in the Flk1⁺/Scl⁺ hemangioblast-containing population found in day-4 embryoid bodies, the cell masses of differentiated tissues formed by mouse ES cells (FIG. 7a). To test if mouse lycat is sufficient to drive differentiation of the endothelial and hematopoietic cell fates, we studied the effect of lycat overexpression in wild type mouse ES cells and an Scl reporter ES cell line (Chung et al., Development 129:5511, 2002). ES cell lines were modified to overexpress mouse lycat utilizing the chicken β-actin or flk1 promoter (FIG. 5) (Hadjantonakis et al., Nature Genet. 19:220, 1998; Wu et al., Mol. Cell. Biol. 23:5680, 2003). The transgene activity in the ES cell lines leads to a significant increase in lycat expression (FIG. 7b). These ES cell lines were differentiated in vitro to generate embryoid bodies (EBs) that contain multiple hematopoietic and endothelial populations. Embryoid bodies were dissociated and subjected to flow cytometry for the endothelial marker Flk1, and the hematopoietic markers Sca1, C-Kit, and CD45 (Chung et al., Development 129:5511, 2002; Spangrude et al., Science 241:58, 1988). As expected of a gene that drives both hematopoietic and endothelial lineages, lycat overexpression causes a two-fold increase of Flk1⁺ cells (FIG. 9 c-g), and a five-fold increase of CD45⁺ cells (FIG. 9 h-l). Thus, it appears that lycat acts upstream of Flk1 and Scl, the genes that were previously thought as acting earliest in these lineages (Huber et al., Nature 432:625, 2004; Schlaeger et al., Development 121:1089, 1995; Shivdasani et al., Nature 373:432, 1995; Robb et al., Proc. Natl. Acad. Sci. U.S.A. 92:7075, 1995).

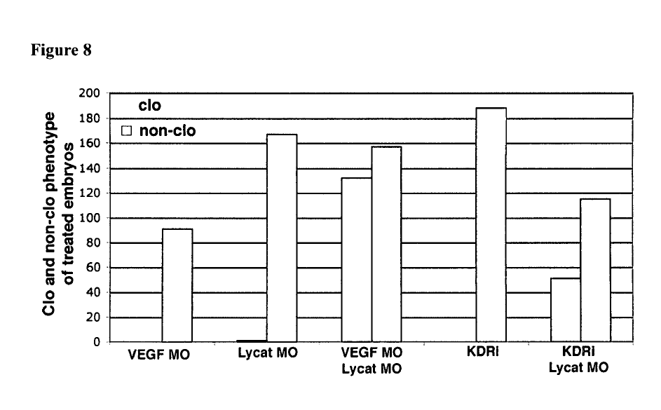

Lycat encodes a novel transmembrane acyltransferase with a C-terminal ER retention signal. Based on synteny and protein sequence homology, we have also identified a single homolog or ortholog of fugu cloche (fugu lycat), mouse cloche (mouse lycat), and human cloche (human lycat) in the vertebrate genomes. The zebrafish lycat protein has identity to human lycat (58%), mouse lycat (50%), and fugu lycat (56%). Interestingly, two acyltransferases called porcupine (Porc) and skinny hedgehog (Ski) were recently isolated in fly. Both Porc and Ski are transmembrane acyltransferase and are localized in the ER. They can function as an acyltransferase for addition of palmitate to wingless and hedgdog, respectively (Nusse, Development 130:5297, 2003; Nybakken and Perrimon, Curr. Opin. Genet. Dev. 12:503, 2002). Therefore we hypothesize the lycat protein also functions as an acyltransferase for lipid modification of vascular endothelial growth factors and other factors. Only VEGF-A, not other isoforms, was demonstrated to be required for both angioblasts and hematopoietic stem cell formation in mice (Carmeliet et al., Nature 380:435, 1996; Ferrara et al., Nature 380:439, 1996). In addition, VEGF189 is a membrane or extracellular-bound protein that is very similar to Wnt and SHH proteins (Houck et al., J. Biol. Chem. 267:26031, 1992; Nusse, Development 130:5297, 2003; Plouet et al., J. Biol. Chem.272:13390, 1997). To test if lycat interacts with VEGF we further took advantage of anti-sense morpholino technology in zebrafish to knock down both VEGF and clo. 84% of embryos injected with combinations of 1 mM VEGF morpholino (VEGF MO) and 0.2 mM lycat morpholino (lycat MO) give rise to clo mutant phenotypes, although no embryos injected with 1 mM VEGF MO and 0.6% embryos injected with 0.2 mM lycat MO have clo phenotypes (FIG. 8). In addition, 76% embryos injected with 0.2 mM lLycat MO and then incubated with 6 μM A676475 (KDRi, a KDR tyrosine kinase inhibitor, Calbiochem), for 24 hours gave rise to clo phenotypes but no embryos incubated only with 6 μM KDRi had clo phenotypes (FIG. 8). The VEGF mRNA expression is relatively normal in clo mutant embryos but VEGFR2 mRNA in clo mutants is about 20% of that in wild-type embryos using quantitative RT-PCR assays. These data show that clo may, at least partly, modulate VEGF protein activity in zebrafish.

Lycat appears to define a key step in the generation of both early blood and vascular cell lineages, lending credence to the notion of a shared embryonic hemangioblastic progenitor. There is accumulating evidence that this lineage is not peculiar to the early embryo, but may also be present in the adult bone marrow (Bailey et al., Blood 103:13, 2004; Grant et al., Nat. Med. 8:607, 2002). The target of lycat acylation remains to be determined, although in Drosophila the acyltransferases, skinny hedgehog and porcupine, are critical to generate fully functional forms of the signaling proteins, Hedgehog and Wingless, respectively (Nusse, Development 130:5297, 2003). Because lycat defines and directs both vessel and blood cell fates and persists ih Sca1⁺/C-Kit⁺ hematopoietic cells in the adult bone marrow, it can be used in the therapeutic reconstitution of these lineages in humans.

In summary, our results from the transgenic GFP labeling experiments have substantiated that lycat is required for the formation of the endocardium, as well as endothelial and hematopoietic stem cells. We have isolated the long-sought clo gene from the lycat genetic interval using a positional cloning approach in zebrafish. The do gene encodes a novel transmembrane, ER-associated protein with a highly conserved acyltransferase enzymatic domain for lipid modification. The do protein may modulate VEGF and other protein activities to specify the hemangioblasts. Clo may modulate a membrane-bound protein such as VEGF 189. We anticipate that the isolation of do may offer us an entry point to dissect signaling mechanisms involved in hemangioblast cell specification and differentiation, as well as to identify therapeutic targets for human ischemic heart diseases and cancers.

TABLE 1
Statistics of lycat morpholino and mRNA injections

| | clo | Wild-type |
|---|---|---|
| | (phenotype) | (phenotype) |
| a. 0.5 mM a345 | 0 | 65 |
| b. 0.3 mM d522 | 132 | 18 |
| c. 0.3 mM d522 + | 33 | 36 |
| 0.3 μg/μl Lycat RNA | | |
| d. 0.3 μg/μl zebrafish | 56 | 35 |
| lycat RNA | | |

a. The control morpholino a345 was dissolved in egg water at 0.5 mM and was injected into 1- to 2-cell stage Tg(flk1:GFP) embryos. None of the 65 injected embryos exhibit the cloche phenotype at 36 hpf and 48 hpf. This represents one of five independent experiments.
b. The zebrafish lycat morpholino d522 was dissolved in egg water at 0.3 mM and 5 nl was injected into 1- to 2-cell stage Tg(flk1:GFP) embryos. 132 out of 150 (88%) injected embryos showed a clear cloche phenotype, i.e., without flk1⁺/GFP⁺ endocardium, blood vessels and visible blood cells at 36 hpf and 48 hpf. 18 embryos showed mild defects in blood vessels and blood cells but had flk1⁺/GFP⁺ endocardium, which were scored as wild-type. This represents one of five independent experiments.
c. Zebrafish lycat morpholino d522 (0.3 mM) and lycat RNA (0.3 mg/ml) were co-injected into 1- to 2-cell stage Tg(flk1:GFP) embryos. 36 out of 69 injected embryos (52%) had no do phenotype at 36 hpf. This represents one of three independent experiments.
d. Zebrafish lycat RNA (0.3 mg/ml) was injected into embryos from clo^{fv087b/+}; gatal:GFP^{tg/tg} sibling crosses, and 35 out of 91 (38.5%) genotypical homozygous cloche embryos showed an increased gatal:GFP signal in the ICM. Genotyping was done by PCR using marker D (FIG. 5a). This represents one of three independent experiments.

### Methods

### Wild-Type, Transgenic GFP Zebrafish and Clo

The cloche alleles, clo^{m39} and clo^{m378} have been previously described (Stainier et al., Development 121:3141, 1995; Stainier et al., Development 123:285, 1996). A new ENU-induced allele, clo^{fv087b} was also used. Genetic mapping of cloche was carried out using hybrid F₁ (Clo^{m39}×TL, clo^{m378}×TL, clo^{fv087b}×WIK). Transgenic lines Tg(flk1:GFP) and Tg(gata1:GFP) (Long et al., Proc. Natl. Acad. Sci. U.S.A. 94:6267, 1997; Cross et al., ArterioScler. Thromb. Vasc. Biol. 23:911, 2003), were crossed with heterozygous clo^{fv087b} fish to generate transgenic clo^{fv087b} individuals. Examining cloche phenotypes and the GFP expression of embryos from sibling crossings identified heterozygous clo^{fv087b/+} fish with the Tg(flk1:GFP) or Tg(gata1:GFP).

Morpholinos and mRNA Injections

Four morpholinos were synthesized: three anti-sense morpholinos targeting the zebrafish lycat ATG site (atg): AACACACACCACGAGGAGACACCAT (SEQ ID NO:5); the second splicing donor site (d322): TAAGCTCTGCGTACCACAGGTAAG (SEQ ID NO:6); the third splicing donor site (d522): CTGAACACACACACTGACCGAAGC (SEQ ID NO:7); and a control morpholino (a345): GCAGCGGGCACTGCTGGTGGAAGT (SEQ ID NO:8) (Gene Tools, LLC). Zebrafish lycat and mouse lycat were cloned into the pCS2 vector, and capped mRNA was synthesized using mRNA in vitro transcription kits (Ambion Inc., Austin, Tex.). Morpholinos and mRNA were injected into one- or two-cell stage wild-type, clo^{fv087b}; Tg(flk1:GFP) or clo^{fv087b}; Tg(gata1:GFP) embryos. Injected embryos were incubated at 28.5° C. for examination of phenotypes or were fixed for in situ hybridization. The bright-field pictures were taken using a Zeiss M12 dissecting microscope, and the fluorescence pictures were taken with a Zeiss Stemi SVII Apo fluorescence microscope.

### RNA In Situ Hybridization and Immuno-Staining

In situ hybridization and antibody staining were done in the presence of blocking solution (Roche, Basil, Switzerland), in the RNA hybridization solution, and antibody staining solution (Schulte-Merker, Looking at embryos, 2002). Antisense flk1 and Scl RNA probes were used for in situ hybridization (Fouquet et al., Dev. Biol. 183:37, 1997; Liao et al., Development 124:381, 1997; Liao et al., Genes Dev. 12:621, 1998).

### Transient Expression of Lycat-Gal4VP16: UAS-GFP

A 1742 bp segment upstream of the start ATG of the zebrafish lycat gene was amplified by PCR (Roche Fast Start Taq polymerase) from genomic DNA (primers 5′-GATGTGTCCGAGAACACGCGTCTGAC-3′ (SEQ ID NO:9) and 5′-CCTCAGCCTGACACACACACAC-3′ (SEQ ID NO:10). The PCR product was subcloned into a modified, promoterless Gal4-VP16 vector (Koster et al., Dev. Biol. 223:329, 2001). The zebrafish lycat-GAL4VP16 plasmid was co-injected with a UAS-GFP construct into one-cell stage embryos and GFP fluorescence was examined at various developmental stages.

### RT-PCR

Wild-type TL embryos were collected at different time points. Mouse ES cell and embryoid bodies were generated, collected, and/or sorted where indicated. Total RNA was isolated using Trizol reagents (Invitrogen). First strand cDNA was synthesized using the Superscript II RT system (Invitrogen). Semi-quantitative PCR was done with Qiagen Taq polymerases and quantitative PCR was done with TaqMan or SYBR Green probes for zebrafish lycat, flk1, and β-actin, as well as mouse lycat and Gapdh using the 7000 Sequence Detection System (ABI Prism).

### Mouse Embryonic Stem Cell Culture and FACS

The tissue culture conditions for mouse ES cells and embryoid bodies and FACS were essentially as previously described (Faloon et al., Development 127:1931, 2000; Wiles et al., Development 111:259, 1991). Mouse R1 ES cells were transfected with an expression construct flk1-mouse lycat-PGKneo or a control construct without mouse lycat cDNA. G418-resistant ES cell clones were isolated and genotyped using PCR with transgene-specific primers. Stable transgenic ES cells were differentiated into day 4 embryoid bodies (EBs) in a suspension EB culture medium. Endothelial and hematopoietic lineages were analyzed by flow cytometry using anti-CD45-Cy5 (Jackson Lab), anti-Flk1-PE, anti-Sca1-PE, and anti-C-Kit-APC antibodies (BD Pharmingen), as previously described (Chung et al., Development 129:5511, 2002). For the purification of Flk1 and Scl cells, the Scl:humanCD4 knock-in ES cell line was utilized (Chung et al., Development 129:5511, 2002). The signal deficient human CD4 is driven by the host endogenous Scl locus and CD4⁺ cells (indicating Scl⁺) were sorted with an antibody to hCD4 (Caltag).

### B. Molecular Interaction of Mouse Lycat and VEGF-A Proteins

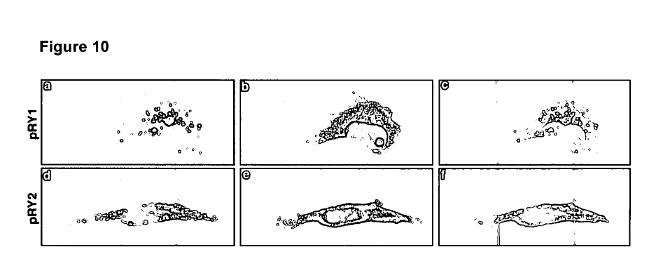

Mouse lycat protein contains KKNE and the zebrafish lycat protein contains DKQE signal in the C-terminals. In order to determine if the mouse lycat protein locates in the ER by expressing GFP-mclo fusion proteins in COS7 cells, we have generated constructs directing expression of a mouse lycat-GFP fusion (C-terminal fusion) called pRY1 (mouse lycat-GFP), and a GFP-mouse lycat fusion (N-terminal fusion) called pRY2(GFP-mouse lycat) under control of the CMV promoter in COS7 cells. Subcellular localization was determined using immuno-staining with the mitochondria marker Cytochrome C (CytC) antibody, the early endosome autoantigen 1 (EEA1) antibody, and the ER marker protein disulphide isomerase (PDI) antibody. pRY2(GFP-mouse lycat) is located in the rough ER and especially concentrated at the intermediate compartment (FIGS. 10d-f). pRY1(mouse lycat-GFP) is likely to be mislocalized due to masking of the ER localization signal by fusion of GFP at the C-terminal of mouse lycat (FIGS. 10a-c). Therefore, zebrafish/mouse lycat is an ER protein, but its acyltransferase activity remains to be determined. In addition, we have also demonstrated that combinations of lycat and VEGF morpholinos sensitize to give rise to do phenotypes (FIG. 8). These data show that VEGF may be one of clo/mouse lycat targets, and that clo/mouse lycat may function as an acyltransferase in the ER to modulate VEGF/Flk1 and other signaling for specification of hemangioblasts.

To investigate the biochemical interaction of clo/mouse lycat with other important vascular and hematopoietic proteins, we have generated polyclonal antibodies against zebrafish lycat, VEGF-A, and mouse lycat using a commercial service (BioSource, MA). Peptides, each 18 amino acids in length, were designed and synthesized. Two rabbits were immunized with each peptide, bled, and subjected to affinity purification. We have received about 120 mg of each affinity-purified antibody for each antigen from BioSource, MA. Antibodies can be characterized using immuno-precipitation and western blots from extracts of zebrafish and mouse embryos, as well as endothelial cell lines C166 and d4T, and using immuno-histochemical staining with embryo or adult tissue sections (Ausubel et al., Current Protocols in Molecular Biology, Vol. 2, 1994). One anti-Lycat is generated as show in FIG. 9c.

Above, we have shown that lycat is required for the formation of hemangioblasts in zebrafish embryos. Heterozygous VEGF knockout mouse embryos were shown to have reduced angioblasts and hematopoietic stem cells (Carmeliet et al., Nature 380:435, 1996; Ferrara et al., Nature 380:439, 1996). Using a whole-mount RNA in situ analysis, we have found lycat mRNA expression in the notochord of zebrafish embryos at 24 hpf and 36 hpf. Combinations of the antisense lycat and VEGF morpholinos sensitize to produce the lycat mutant phenotypes, indicating that lycat may modulate VEGF activities at the protein level, since there is normal expression of VEGF mRNA in do mutant embryos. Therefore, lycat acyltransferase activities can be tested for palmitoylation of VEGF.

Lycat protein is a member of the large family of acyltransferases. Clo is required for VEGF/Flk1 signaling. Skinny hedgehog is required for the hedgehog protein processing and palmitoylation, and porcupine is required for the wingless protein processing and possibly palmitoylation (Chamoun et al., Science 293:2080, 2001; Kadowaki et al., 1996; Stainier et al., Development 121:3141, 1995). There were DWnt-3/5 and DWnt1protein mis-localization in porcupine embryos (Tanaka et al., J. Biol. Chem. 277:12816, 2002; van den Heuvel et al., EMBO J. 12; 5293, 1993; Zhai, et al., J. Biol. Chem. 279, 33220, 2004). Using immuno-histochemical staining, VEGF protein is located in developing wild-type and do embryos. The expression domains of VEGF are compared with that of the lycat protein in developing embryos and the kidney marrow. Any changes in the subcellular localization of VEGF in the wild-type and clo embryos is measured.

The heparin-binding VEGF isoforms (VEGF189 and VEGF165) were reported to be membrane bound to cell surface or extracellular matrix in tissue cultured cells over-expressing VEGF (Houck et al., J. Biol. Chem. 267:26031, 1992; Plouet et al., J. Biol. Chem. 272:13390, 1997). The mouse VEGF189 cDNA is subcloned into mammalian expression vector pCX where the VEGF gene is under control of the chicken β-actin promoter (Hadjantonakis et al., Nature Genet. 19:220, 1998). A PGK-neo cassette is inserted into the expression vector for G418 selection. To produce stable cells expressing VEGF, NIH3T3 cells are used (Xiong et al., Dev. Dyn. 212:181, 1998). A stable cell population is selected by 0.5-1.0 mg/ml of G418.

To determine if VEGF has palmitate modification in mammalian cells, metabolic labeling of VEGF by ³H-palmitate or labeling palmitoylation sites by ³H—N-Ethylinaleimide (³H-NEM) is used (Drisdel and Green, BioTechniques 36:276, 2004). ³⁵S methionine/cysteine labeling is included as a control for protein synthesis. NIH3T3 cells and stable NIH3T3 cells expressing VEGF are cultured for metabolic labeling with ³⁵S methionine/cysteine and ³H-palmitate, respectively. ³H-labelled proteins are run on SDS-PAGE and transferred to nitrocellulose membranes. Gels contained ³⁵S-labeled proteins are stained, fixed, treated with Amplify™ (Amersharn Biosciences, USA), for 30 minutes, dried, and exposed to films at −70° C. with intensifying screen. A new method of labeling palmitoylation was recently developed based on the susceptibility of the fatty acylthioester bond to neutral hydroxylamine cleavage, resulting in removal of the fatty acid and generation of a free sulfhydryl. Subsequently, sulfhydryl-specific reagents such as ³H-NEM is used to label palmitoylation sites quantitatively. NIH3T3 cells or stable NIH3T3 cells expressing VEGF are solubilized, and the resulting lysates are immunoprecipitated with anti-VEGF antibody. The resulting protein-antibody complex is treated with 1 M hydroxylamine, and labeled with ³H-NEM. Labeled protein samples are resolved on SDS-PAGE and transferred to nitrocellulose membranes. With the combination of these two approaches, palmitoylation of VEGF can be detected.

### Methods

### Generating Polyclonal Antibodies

Polyclonal antibodies against zebrafish lycat, VEGF-A, and mouse lycat protein were generated by using a commercial service from BioSource, MA. Mouse monoclonal antibodies against VEGF-A are commercially available from Pharmingen, Inc. and Santa Cruz Biotechnology, Inc. They are suitable for immuno-precipitation, western blotting, and immuno-histochemical staining.

### RNA In Situ Analysis and Immunohistochemical Staining of Embryos and Tissue Sections

Standard protocols are for immunohistochemical staining are used (Nagy et al., Manipulating the mouse embryo, 2003; Schulte-Merker, Looking at embryos, 2002; Xiong et al., Dev. Dyn. 212:181, 1998; Xiong et al., Dev. Biol. 206:123, 1999).

### Metabolic Labeling

NIH3T3 cells are maintained in DMEM (GibcoBRL) supplemented with 10% calf serum (CS) (Hyclone). Cell cultures are incubated in methionine-free DMEM for 15 minutes and labeled in methionine-free DMEM containing 100 μCi/ml of ³⁵S methionie/cystein (EXPE³⁵S³⁵S™; PerkinElmer, Boston, Mass.), for 1 hour as described (Drisdel and Green, BioTechniques 36:276, 2004). Cells are then washed twice with DMEM supplemented with 5 mM cold methionine and incubated at 37° C. in culture medium for the duration of the chase. For ³H-palmitate labeling, palmitic acid [10, 11-³H] (10 mCi/ml; American Radiolabeled Chemicals, St. Louis, Mo., USA) is used. DMEM containing 1% CS is added to the vial, vortex mixed vigorously, and diluted to a final activity of 100 μCi/ml. This labeling medium (1 ml/6-cm plate) is added to cell cultures and incubated for 0.5-4 hours at 37° C. ³H-labelled proteins are run on SDS-PAGE and transferred to nitrocellulose membranes. Gels contained ³⁵S-labeled proteins are stained, fixed, treated with Amplify™ (Amersham Biosciences, USA), for 30 minutes, dried, and exposed to films at −70° C. with intensifying screen.

³H—N-Ethylmaleimide labeling

Cell lysates are prepared from NIH3T3 cells and NIH3T3 cells expressing VEGF protein, and are incubated with Sepharose 4B to remove proteins that bound nonspecifically to the Sepharose. Samples are centrifuged, and the supernatants are rotated overnight with anti-VEGF antibody. The protein-antibody complex is precipitated with Protein G-Sepharose (Amersham Biosciences). NEM [ethyl-1,2-³H] (³H-NEM) (1 mCi/ml; PerkinElmer, USA), supplied in pentane is used. Immunoaffinity purified protein bound to Protein G-Sepharose is treated with 1 M hydroxylamine, pH 7.4, for 1 hour at room temperature. Controls are incubated in the presence of 1 M Tris, pH 7.4. The beads are washed twice with lysis buffer plus 1% Triton X-100. The beads are resuspended in 8 volumes of lysis buffer containing 0.5 μM ³H-NEM and rotated at room temperature for 3 hours. Samples are resolved on SDS-PAGE and transferred to nitrocellulose membranes. Immunoblots are performed with anti-VEGF antibody. After blotting, membranes are stripped, dried, and exposed to a ³H-specific phosphor screen for 24 to 72 hours. Screens are scanned on a PhosphorImager (Zhai et al., J. Biol. Chem. 279, 33220-33227, 2004).

### C. Isolating the Mouse Lycat Gene and Generating Mouse Lycat Null ES Cells and Mice

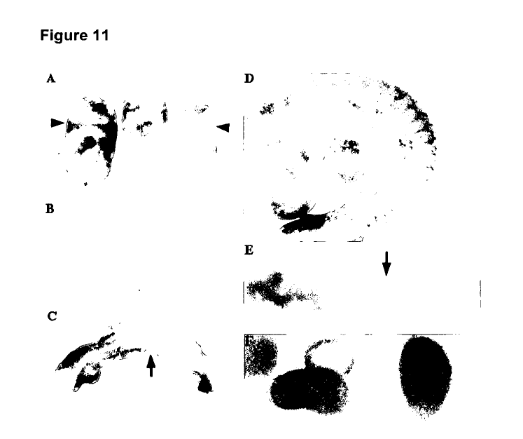

A single mouse homolog (mouse lycat) was identified to be highly homologous to the zebrafish lycat using NCBI Blast Searching. Two sequences were found to contain the full length coding sequence of mouse lycat. Based on the sequence data, a full length mouse lycat cDNA clone was isolated and cloned using RT-PCR with E8.5 embryo RNA. Using whole-mount RNA in situ analysis, we have detected very weak mouse lycat expression in the heart and aorta of embryos at 8.5 dpc and 9.5 dpc (FIGS. 11e and 11f). Vascular-specific gene Egf17 was used as a positive control for RNA in situ analysis (FIGS. 11a-11d). This indicates that mouse lycat may also be a vascular-specific acyltransferase. We also evaluated lycat mRNA expression using RT-PCR and found that mouse lycat has low-abundant expression in liver, brain, heart, lung, prostate, salivary gland, smooth muscle, spleen, stomach, testis, thymus, and uterus; as well as in embryos at 7.5, 9.5, 11, 15, and 17 dpc (FIG. 9a). For RT-PCR, 1 μg of the total RNA was used to synthesize the first strand of cDNA, and one tenth of each RT reaction was then used as a PCR template with 40 PCR cycles of amplification. The PCR fragments were resolved in 1% agarose gel.

To make a gene-targeted construct in the mouse lycat locus, we first determined a mouse lycat genomic sequence using the mouse lycat cDNA sequence as a query to perform a Blast Search against the mouse genomic sequence databases. Five BAC clones containing the mouse lycat gene were identified and requested through the Children's Hospital Oakland Research Institute. To define mouse lycat gene function in ES cells and mice, we have started to generate heterozygous and homozygous null mouse lycat ES cell clones. In brief, one BAC clone containing the mouse lycat locus was used as a template to amplify the 5′ arm (5950 bp in length), containing the first and second introns and the second coding exon, and the 3′ arm (5320 bp in length), containing the third exon and intron using the Expand Long Range PCR System (Roche). A gene-targeted construct was made to contain the 5′ arm, a promoter-less LacZ reporter gene, a PGK-neo cassette, and the 3′ arm. Homologous recombination between the targeting vector and the genomic DNA is expected to disrupt the mouse lycat gene and place LacZ under control of the endogenous mouse lycat gene promoter. Linearized gene-targeting constructs were electroporated into R1 ES cells (Nagy and Rossant, Gene targeting:a practical approach, 147, 1993; Xiong et al., Dev. Dyn. 212:181, 1998), followed by G418 selection for about 10 days. Approximately 400 G418-resistant ES cell clones were isolated and expanded, and ES cell DNA was isolated as described (Xiong et al., Dev. Dyn. 212:181, 1998). Southern analysis of ES cell DNA, digested with SacI or EcoRV, is performed to identify the expected homologous recombinant event. Clo143 and clo145 are the 5′ and 3′ flanking genomic DNA probes outside of the homologous regions and between the gene-targeting vector and endogenous genomic DNA. The flanking probes are used to distinguish the length of the polymorphisms between wild-type and mutant alleles. Southern analysis can be performed to identify heterozygous (mouse lycat/+) ES cell clones using clo143. Mouse lycat-null ES cell clones are generated by selecting heterozygous mouse lycat ES cells in high concentrations of G418 (Mortensen et al., Mol. Cell. Biol. 12:2391, 1992), and examining their DNA by Southern analysis. At least two homozygous ES cell clones will be isolated for further experiments.

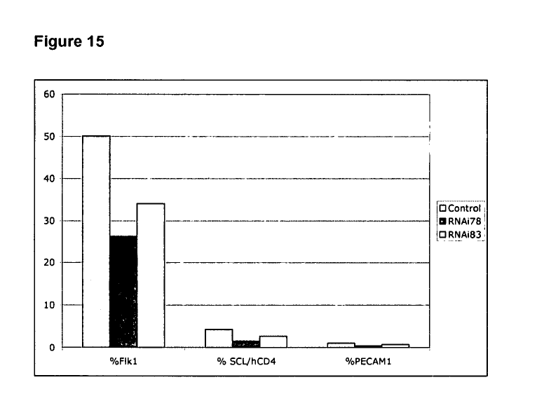

We have also tested the mouse lycat function in hemangioblast formation during ES cell differentiation into EBs using an RNAi approach. Two independent mouse lycat RNAi constructs (RNAi78 and RNAi83) were made so that the RNAi is under control of the U6 promoter (Sui et al., Proc. Natl. Acad. Sci. U.S.A. 99:5515, 2002). The mouse lycat RNAi construct DNA was introduced into hCD4_Sclknock—in ES cells (hCD4_Scl ES) (Chung et al., Development 129:5511, 2002). The hCD4_Scl ES cells, were generated by knock-in of the non-functional human CD4 receptor into the Scl locus (ibid). This allows for fluorescence activated cell sorter (FACS) analysis with the hCD4 antibody to quantitatively count Scl+ cells. One tenth of a PGK-neo construct was co-transfected with the RNAi construct DNA for G418 selection. ES cells were selected with G418 for 10 days. The control ES and G418+ ES cells containing RNAi were differentiated into EBs in a suspension culture (Faloon et al., Development 127:1931, 2000). For fluorescence activated cell sorter (FACS) of FLK1+, CD4+ (=Scl+), and PECAM1+ cells, biotinylated mouse anti-human CD4 monoclonal antibodies (CALTAG), phycoerythrin-conjugated anti-FLK1 monoclonal antibodies, and FITC anti mouse PECAM1 antibodies (BD Biosciences, San Jose, Calif.) were used (ibid). Our preliminary results showed that in day three embryoid bodies (EBs), both mouse lycat RNAi78 and RNAi83 had inhibitory effects on the Flk1+/Scl+ cells, as well as the PECAM1+ endothelial cells (FIG. 15). Therefore, mouse lycat may play a similar and important role in the formation of hemangioblasts.

By using the mouse ES cell differentiation system, blast-colony-forming cells (BL-CFC) or Flk1+/VE-caherin+ cells were isolated and proposed to be equivalent to hemangioblasts (Choi et al., Development 125:725, 1998; Faloon et al., Development 127:1931, 2000; Kennedy et al., Nature 386:488, 1997; Nishikawa et al., Development 125:1747, 1998).

Gene-targeting in ES cells and in mice is widely applied for defining mammalian gene function (Nagy et al., Manipulating the mouse embryo, 2003). Southern analysis of ES cell DNA, digested with SacI or EcoRV, is designed to identify the expected homologous recombinant event, using the flanking genomic DNA probes outside of the homologous regions between the gene-targeted vector and the endogenous genomic DNA.

To generate knock-out mouse lines that carry a mouse lycat null allele, two independent ES cell clones with a targeted mouse lyeat allele are selected for generating germ-line chimeras by microinjecting ES cells into blastocysts with the help of the MGH ES Cell/Knockout Facility. Alternatively, germ-line chimeras are generated by using the ES cell-diploid embryo aggregation method as the PI has done previously (Nagy et al., Proc. Natl. Acad. Sci. U.S.A. 90:824, 1993; Xiong et al., Dev. Dyn. 212:181, 1998). To obtain germ-line transmission, chimeric males are bred with C57BL/6J females, and tail DNA of the F1 agouti offspring is examined for the presence of the targeted mouse lycat allele. Heterozygous F1 males and females are crossed to obtain homozygous offspring. If no viable heterozygous mice are obtained, completely ES cell-derived embryos are generated by using the mouse lycat-null ES cell-tetraploid embryo aggregation approach as described previously (Carmeliet et al., Nature 380:435, 1996; Nagy et al., Proc. Natl. Acad. Sci. U.S.A. 90:824, 1993; Xiong et al., Dev, Dyn. 212:181, 1998). This allows us to examine the function of the mouse lycat gene during mouse cardiovascular and blood development without establishing transgenic knockout mouse lines.

As an alternative approach, the LacZ reporter gene expression is used to define mouse lycat mRNA expression during early mouse embryogenesis. The knock-in LacZ is placed under the control of the endogenous mouse lycat promoter. To label and isolate the mouse lycat+ hemangioblasts, a EGFP reporter gene is introduced into the 3′UTR of the mouse lycat. The EGFP+ hemangioblasts, generated in embryoid bodies and in transgenic embryos, are then be isolated using FACS analysis (Faloon et al., Development 127:1931, 2000).

Alternatively, a conditional mouse lycat allele with flanking loxP sites (Mclo^{+/loxP}) can be developed. Ablation of mouse lycat at approximately 8.0 pdc is obtained by crossing Tie2-Cre transgenic mice with homozygous loxP alleles (Mclo^{loxP/loxP}) (Kisanuki et al., Dev. Biol. 230:230, 2001).

### Methods

### Mouse ES Cell Culture Manipulation of Mouse Embryos, RNA In Situ Hybridization, and Histology

Protocols, for use in mouse ES cell culture, were described in detail previously (Nagy et al., Manipulating the mouse embryo, 2003; Xiong et al., Dev. Dyn. 212:181, 1998; Xiong et al., Dev. Biol. 206:123, 1999).

Generation of Germ-Line Chimeras by Microiniecting ES Cells into Blastocysts

A protocol for generation of germ-line chimeras is described in the book “Manipulating the mouse embryos” (Nagy et al., Manipulating the mouse embryo, 2003).

### Generation of Diploid and Tetraploid Aggregation Chimeras

A protocol for generation of aggregation chimeras was modified based on the original method (Nagy et al., Proc. Natl. Acad. Sci. U.S.A. 90:824, 1993) and was previously described (Xiong et al., Dev. Dyn. 212:181, 1998).

### D. Over or Ectopic Expression Constructs of Mouse Lycat

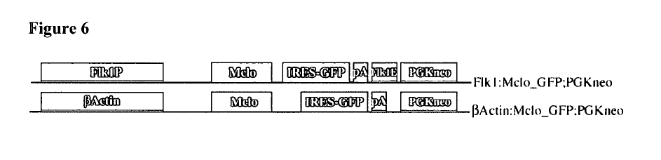

We generated expression constructs directing mouse lycat and GFP expression under control of the mouse Flk1 promoter and enhancer, and chicken β-Actin promoter and CMV-IE enhancer, respectively (FIGS. 6a and 6b). A PGKneo cassette was inserted into both constructs designed for G418 selection in transfected mouse ES cells (FIGS. 6a and 6b). To make an over-expression construct, we placed mouse lycat cDNA-SV40 poly A and an IRES-GFP cassette under the control of the mouse Flk1 promoter/enhancer that forms the Flk1:Mclo_IRESgfp construct. We then inserted a PGK-neo cassette into the Flk1:Mclo_IRESgfp construct to generate the final construct Flk1:mclo_IRESgfp;PGKneo (FIG. 6a). The mouse Flk1 promoter and enhancer elements were used to direct reporter LacZ gene expression exclusively in the hemangioblasts and vascular endothelial cells in mouse embryos (Wu et al., Mol. Cell. Biol. 23:5680, 2003). We have obtained the Flk1 promoter/enhancer DNA clone from Dr. Cam Patterson at University of North Carolina-Chapel Hill. To make an ectopic expression construct, we placed mouse lycat cDNA-SV40 poly A and an IRES-GFP cassette under control of the chicken β-actin promoter and CMV-IE enhancer to form the construct βActin:mclo_IRESgfp, and we inserted a PGK-neo cassette into the βActin:Mclo_IRESgfp construct to yield the final construct βActin:Mclo_IRESgfp;PGKneo (Hadjantonakis et al., Nature Genet. 19:220, 1998). This promoter/enhancer has been successfully used to generate a ubiquitous EGFP expression in transgenic ES cells and mice (ibid). We have obtained a DNA clone of the chicken β-actin promoter and CMV-IE enhancer from Dr. Angus Nagy at Mount Sinai Hospital, Toronto.

To generate transgenic mouse lines, the linearized expression construct DNA was microinjected into the pronuclei of C57BL/6 mouse zygotes. F₀ founders were established by using a commercial core service from the Transgenic Mouse Facility of Cutaneous Biology Research Center (CBRC) at MGH. Founders were screened for the presence of the transgene in their tail DNA by using PCR with transgene-specific primers. We have found that 11 out of 27 for Tg[Flk1:Mclo_IRESgfp], and 17 out of 35 for Tg[β-Actin:Mclo_IRESgfp] founders contain the transgene in their tail DNA. Transgene copies of each founder are determined using Southern analysis of tail DNA. Five transgenic founders (F₀) of each line are bred to establish F1 transgenic lines. We have identified 18 males and 7 females of transgenic Tg[Flk1:Mclo_IRESgfp] mice out of 107 mice from F₀ outcross with CD1, as well as 4 males and 1 female of transgenic Tg[β-Actin:Mclo_IRESgfp] mice out of 46 mice from F₀ outcross with CD1. In addition, 5 pregnant mice from Tg[β-Actin:Mclo_IRESgfp] F₀ founder outcross did not give birth. These data indicate that ectopic expression of mouse lycat by β-Actin promoter may cause embryonic lethality. We are maintaining the transgene in the hemizygous state to minimize complications caused by insertional mutations.

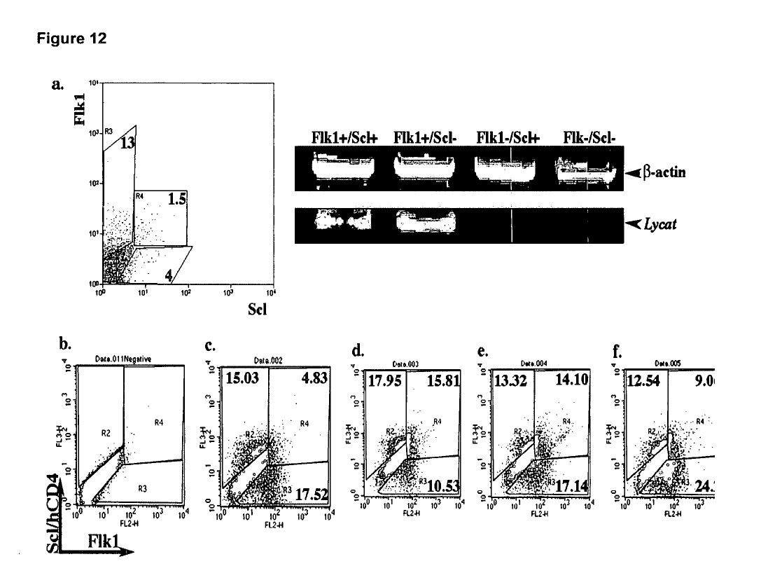

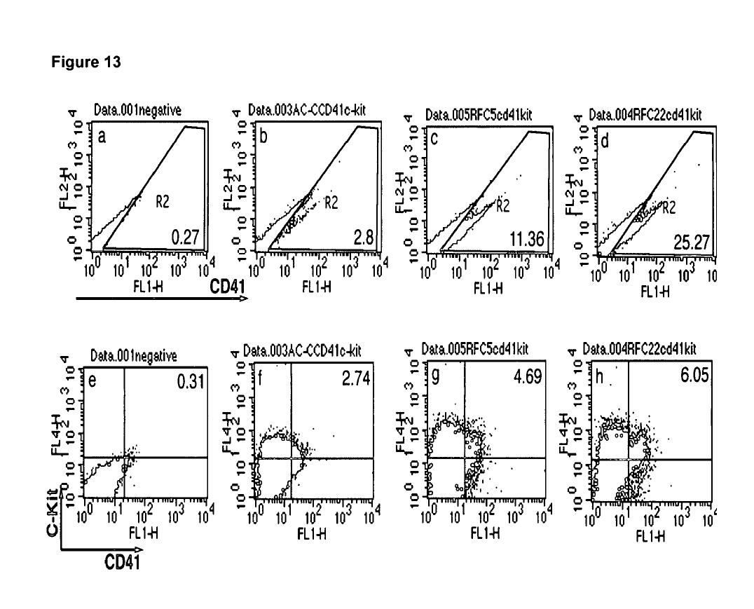

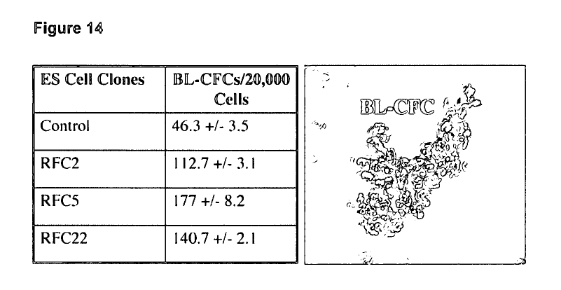

To test if mouse lycat is sufficient to drive differentiation of the endothelial and hematopoietic cell fates, we studied the effect of lycat overexpression in wild type mouse ES cells and an scl reporter ES line (Chung et al., Development 129:5511, 2002). ES lines were modified to overexpress mouse lycat utilizing the chicken β-actin or flk-1 promoter (Chung et al., Development 129:5511, 2002; Nagy et al., Proc. Natl. Acad. Sci. U.S.A. 90:824, 1993; Xiong et al., Dev. Dyn. 212:181, 1998). The transgene activity in the ES cell lines leads to a significant increase in lycat expression (FIG. 7). 24 G418-resistant ES clones of each construct were isolated and expanded. The ES cell DNA was then isolated as described (Xiong et al., Dev. Dyn. 212:181, 1998). ES cell clones containing the transgene were identified by using PCR with the same transgene-specific primers for genotyping tail DNA of mice. 16 stable ES cell clones containing the transgene Tg[Flk1:Mclo_IRESgfp_PGKneo] or Tg[βactin:Mclo_IRESgfp_PGKneo] was isolated. Six control ES cell clones containing Tg[βActin:EGFP] were also isolated. Three independent ES cell clones from each construct were expanded for ES cell differentiation into embryoid bodies (Xiong et al., Dev. Dyn. 212:181, 1998). These ES lines were differentiated in vitro to generate embryoid bodies (EBs) that contain multiple hematopoietic and endothelial populations (Choi et al., Development 125:725, 1998). Embryoid bodies were dissociated and subjected to flow cytometry for the endothelial marker Flk1, and the hematopoietic markers CD41 and CD45 (Spangrude et al., Science 241:58, 1988; Chung et al., Development 129:5511, 2002). As expected of a gene that drives both hematopoietic and endothelial lineages, lycat overexpression causes a two-fold increase of the Flk1⁺ (FIGS. 7d-g), a five-fold increase of CD45⁺ (FIGS. 7i-l), and a two-fold increase of CD41 (FIG. 13). Furthermore, the mouse lycat gene is enriched in the Flk1⁺/Scl⁺ hemangioblast population (FIG. 12a), as well as the mouse lycat transgene leads to a three-fold increases of Flk1⁺/Scl⁺ hemangioblasts (FIG. 12b) by FACS, and blast colony forming cells (BL-CFCs) in methylcellulose culture assays for hemangioblasts (FIG. 14). Thus, it appears that lycat acts upstream of Flk1 and Scl, the genes previously defined as acting earliest in these lineages (Huber et al., Nature 432:625, 2004; Schlaeger et al., Development 121:1089, 1995; Shivdasani et al., Nature 373:432, 1995; Robb et al., Proc. Natl. Acad. Sci. U.S.A. 92:7075, 1995).

### Methods

### Generation of Transgenic Mice

A detailed protocol for generating transgenic mice is found in the book “Manipulating the mouse embryos” (Nagy et al., Manipulating the mouse embryo, 2003). A technical and commercial service will be available in the Transgenic Mouse Facility of CBRC at MGH.

### Fluorescence Activated Cell Sorter (FACS)

For purifying circulating angioblasts, peripheral blood is obtained from the heart immediately before sacrifice, and separated by Histopaque-1083 (Sigma) density gradient centrifugation. Light density mononuclear cells (MCs) are harvested and washed twice with Dulbecco's PBS (DPBS) without calcium. The isolated MCs are incubated in DPBS containing DiI-labeled acLDL. The cells are then incubated with fluorescent-labeled antibodies (mouse Flk1 and CD34) and analyzed by FACS (Asahara et al., Science 275:964, 1997; Faloon et al., Development 127:1931, 2000). ES cell-derived hemangioblasts or BL-CFCs are sorted on a Becton Dickinson flow cytometer for GFP expression and for FLK1 expression (Faloon et al., Development 127:1931, 2000). Sorting of HSC is done as described (Domen et al., J. Exp. Med. 191:253, 2000). In brief, bone marrow cells are enriched for c_Kit+ cells using MACS-columns (Miltenyi Biotechnology). The cells are stained with FITC-19XE5 (anti-Thy1.1), lineage-cocktail, Texas red-E13-161-7 (anti-Sca-1), APC-2B8 (anti-c-Kit), and biotin-3C11 (anti-c-Kit).

All documents cited above are incorporated herein by reference. Other embodiments of the invention are within the scope of the following claims.

What is claimed is:

1-12. (canceled)

13. An isolated nucleic acid molecule encoding lycat.

14. The nucleic acid molecule of claim 13, comprising the sequence of SEQ ID NO:1 or SEQ ID NO:3.

15. A substantially purified protein that is encoded by the nucleic acid molecule of claim 13.

16. (canceled)

17. The isolated nucleic acid molecule of claim 13, wherein the nucleic acid molecule is operatively linked to an expression control sequence.

18. A vector comprising the nucleic acid molecule of claim 13.

19-36. (canceled)

37. A method of inducing the production or development of hematopoietic or endothelial cells in a subject in need thereof, the method comprising administering to the subject an agent that activates expression of lycat, stabilizes lycat message or protein, or increases lycat activity.

38. A method of treating a subject for a proliferative disorder, the method comprising administering to the subject an agent that decreases expression of lycat, destabilizes lycat message or protein, or decreases lycat activity.

39. The method of claim 37, wherein the agent is selected from the group consisting of an expression vector encoding lycat, a protein preparation of lycat, a nucleic acid molecule encoding lycat, an antibody that specifically binds lycat, a nucleic acid molecule comprising an siRNA, and any combination thereof.

40. The method of claim 39, wherein the lycat is human lycat.

41. The method of claim 39, wherein the expression vector is an adeno-associated virus (AAV), an adenovirus, or a retrovirus-based vector.

42. The method of claim 39, wherein the expression vector and the nucleic acid molecule comprise the sequence of SEQ ID NO:1 or SEQ ID NO:3.

43. The method of claim 39, wherein the protein preparation comprises the sequence of SEQ ID NO:2 or SEQ ID NO:4.

44. The method of claim 39, wherein the nucleic acid molecule comprises the siRNA further comprising the sequence set forth in SEQ ID NO:5, 6, 7, or 8.

45. The method of claim 39, wherein the antibody specifically binds to all or a portion of the sequence of SEQ ID NO:2 or SEQ ID NO:4.

46. The method of claim 38, wherein the agent is selected from the group consisting of an antibody that specifically binds lycat and a nucleic acid molecule comprising an siRNA, and a combination thereof.

47. The method of claim 46, wherein the lycat is human lycat.

48. The method of claim 46, wherein the antibody specifically binds to all or a portion of the sequence of SEQ ID NO:2 or SEQ ID NO:4.

49. The method of claim 44, wherein the nucleic acid molecule comprises the siRNA further comprising the sequence set forth in SEQ ID NO:5, 6, 7, or 8.

50. The protein of claim 15, comprising the sequence of SEQ ID NO:2 or SEQ ID NO:4.

51. A cell comprising the vector of claim 18.
